(19) United States
(12) Patent Application Publication
(10) Pub. No.: US 2008/0021085 A1
(43) Pub. Date: Jan. 24, 2008

(54) METHOD OF REDUCING ABETA42 AND TREATING DISEASES

(75) Inventors: Edward Hao Mang Koo, La Jolla, CA (US); Todd Eliot Golde, Ponte Vedra Beach, FL (US); Douglas Roger Galasko, San Diego, CA (US); Sascha Weggen, Duesseldo (DE)

Correspondence Address: MYRIAD GENETICS INC.;INTELLECUTAL PROPERTY DEPARTMENT, 320 WAKARA WAY, SALT LAKE CITY, UT, 84108, US

(73) Assignees: Mayo Foundation for Medical Education and Research, Rochester, MN (US); The Regents of The University of California, Oakland, CA (US)

(21) Appl. No.: 11/766,589

(22) Filed: Jun. 21, 2007

Related U.S. Application Data
(63) Continuation-in-part of application No. 11/170,776, filed on Jun. 28, 2005.
Application No. 11/170,776 is a continuation-in-part of application No. 10/012,606, filed on Dec. 7, 2001, now Pat. No. 6,911,466.
Application No. 10/012,606 is a continuation-in-part of application No. PCT/US01/11956, filed on Apr. 12, 2001.
(60) Provisional application No. 60/196,617, filed on Apr. 13, 2000.

(51) Int. Cl. A61K 31/192; A61K 31/196; A61K 31/403; A61P 25/28

(52) U.S. Cl. 514/411; 514/420; 514/567; 514/569; 514/570; 514/789

(57) ABSTRACT

The invention provides a method of preventing, delaying, or reversing the progression of Alzheimer's disease by administering an Aβ₄₂ lowering agent to a mammal under conditions in which levels of Aβ₄₂ are selectively reduced, levels of Aβ₃₈ are increased, and levels of Aβ₄₀ are unchanged. The invention provides methods and materials for developing and identifying Aβ₄₂ lowering agents. In addition, the invention provides methods for identifying agents that increase the risk of developing, or hasten progression of, Alzheimer's disease. The invention also provides compositions of Aβ₄₂ lowering agents and antioxidants, Aβ₄₂ lowering agents and non-selective secretase inhibitors, as well as Aβ₄₂ lowering agents and acetylcholinesterase inhibitors. The invention also provides kits containing Aβ₄₂ lowering agents, antioxidants, non-selective secretase inhibitors, and/or acetylcholinesterase inhibitors as well as instructions related to dose regimens for Aβ₄₂ lowering agents, antioxidants, non-selective secretase inhibitors, and acetylcholinesterase inhibitors.

### CROSS REFERENCE TO RELATED APPLICATIONS

This application is a continuation-in-part of U.S. application Ser. No. 11/170,776, filed Jun. 28, 2005, which is a continuation-in-part of U.S. application Ser. No. 10/012,606, filed Dec. 7, 2001, now U.S. Pat. No. 6,911,466, which is a continuation-in-part and claims priority of PCT Application PCT/US01/11956, filed Apr. 12, 2001, which claims the benefit of U.S. Provisional Application No. 60/196,617, filed Apr. 13, 2000, all of which are herein incorporated by reference in their entireties.

### STATEMENT AS TO FEDERALLY SPONSORED RESEARCH

Funding for the work described herein was provided, in part, by the federal government, which may have certain rights in the invention.

### BACKGROUND

1. Technical Field

The invention relates to the use of Aβ₄₂ lowering agents to prevent, delay, or reverse the progression of Alzheimer's disease. The invention also relates to methods and materials involved in identifying Aβ₄₂ lowering agents that can be used to prevent, delay, or reverse Alzheimer's disease as well as methods and materials involved in identifying agents that (1) increase the risk of developing or (2) hasten the progression of Alzheimer's disease in a mammal.

2. Background Information

Alzheimer's disease (AD) is the most common form of age-related neurodegenerative illness. The defining pathological hallmarks of AD are the presence of neurofibrillary tangles and senile plaques in the brain. Amyloid β polypeptides (Aβ) are the major constituents of amyloid plaques and are derived from altered processing of amyloid precursor proteins (APPs). Aβ consists predominantly of two forms, Aβ₄₀ and Aβ₄₂. Although Aβ₄₀ is the predominant form, recent evidence suggests that Aβ₄₂ is the pathogenic form. In addition to Aβ₄₀ and Aβ₄₂, the processing of APP generates other Aβ forms such as Aβ₃₉, Aβ₃₈, Aβ₃₇, and Aβ₃₄.

Genetic predisposition is the largest cause of AD in the population, accounting for perhaps 50% or more cases of this disorder (Blacker et al. (1998) Arch Neurol 55:294-6). In the past decade, epidemiological evidence suggests that non-steroidal anti-inflammatory drug (NSAID) treatment, estrogen replacement therapy, and antioxidant therapy may have beneficial effects in AD. Experimental support for these treatment methods, however, is indirect. In addition, there is no convincing evidence from randomized clinical trials that any medication tested to date slows the progression of AD. The rational development of compounds that influence key pathways or targets involved in the development of AD is critically important.

### SUMMARY

The invention relates to the use of Aβ₄₂ lowering agents to prevent, delay, or reverse the progression of AD. The invention is based on the discovery that some but not all NSAIDs can reduce Aβ₄₂ secretion from cells in vitro or in animals, when used at a sufficiently high amount.

Thus, in one aspect, the present invention provides a method for treating, or delaying the onset of, Alzheimer's disease or mild cognitive impairment, which comprises identifying a patient in need of the treatment or delay, and administering to the patient a therapeutically effective amount an Aβ₄₂-reducing agent. For example, an Aβ₄₂-reducing agent can be an NSAID, or a structural derivative or analogue thereof that can reduce Aβ₄₂ secretion from cells in vitro or in animals. In one embodiment, the therapeutically effective amount is an Aβ₄₂-reducing effective amount. In specific embodiments, the Aβ₄₂-reducing agent is administered at an amount sufficient to reduce the level of Aβ₄₂ secretion in an assay performed in any one of Examples 8-38, e.g., Example 8 or 12 or 17.

Preferably, the method for treating, or delaying the onset of, Alzheimer's disease or mild cognitive impairment further includes analyzing a biological marker in a sample obtained from the patient before and/or after the administration of the Aβ₄₂-reducing agent, e.g., suitable NSAIDs or structural derivatives or analogues thereof. The sample can be a plasma or CSF sample, and the biological marker can be selected from isoprostanes, M-CSF, MCP-1, Tau, P-tau181, Aβ₄₂, Aβ₃₈, and Aβ₄₀. In some embodiments, at least one of the following is monitored before and/or after the administration of said Aβ₄₂-lowering agent: (1) the plasma or CSF concentration of Aβ₄₂, (2) the plasma or CSF concentration of Aβ₄₀, (3) the plasma or CSF concentration of Aβ₃₄, Aβ₃₆, Aβ₃₇, or Aβ₃₉, (4) the plasma or CSF concentration of Aβ₃₈, (5) the Aβ₄₂/Aβ₄₀ concentration ratio in plasma or CSF, (6) the concentration ratio between Aβ₄₀ and one of Aβ₃₄, Aβ₃₆, Aβ₃₇, Aβ₃₈ and Aβ₃₉, or a combination thereof, in plasma or CSF, (7) the ratio between the concentration of Aβ₄₂ and Aβ₃₈ in plasma or CSF, and (8) the total concentration of Aβ peptides in plasma or CSF.

Useful NSAIDs or structural derivatives or analogues include an aryl propionic acid or a pharmaceutically acceptable salt or ester or derivative or analogue thereof. Other examples of useful Aβ₄₂-reducing agents include NSAIDs selected from the group consisting of flufenamic acid, fenoprofen, sulindac sulfate, indomethacin, mefenamic acid, and flurbiprofen, and pharmaceutically acceptable salts or esters thereof, and derivatives and analogues thereof. Preferably, the NSAIDs, or structural derivatives or analogues thereof, lack the ability to inhibit COX-1 or COX-2, or both COX-1 and COX-2 enzymatic activity as tested in, e.g., Example 10. A preferred Aβ₄₂-reducing agent is R-flurbiprofen (tarenflurbil, (−)-(2R)-2-(2-fluorobiphenyl-4-yl)propanoic acid) or a pharmaceutically acceptable salt or ester or derivative thereof.

In some embodiments, the method of the present invention further comprises administering to the patient an acetylcholinesterase inhibitor, or an antioxidant.

In preferred embodiments, the Aβ₄₂-reducing agents useful for treating AD are those that can selectively reduce the level of the pathogenic Aβ₄₂ form, do not affect the level of Aβ₄₀, and increase levels of Aβ forms smaller than Aβ₄₀ such as Aβ₃₈. More specifically, the invention provides methods and materials related to identifying Aβ₄₂ lowering agents, including NSAIDs, NSAID derivatives, and NSAID analogues, that (1) can reduce the level of Aβ₄₂ by reducing APP processing into Aβ₄₂ or by increasing Aβ₄₂ catabolism; (2) increase the level of Aβ₃₈ by increasing APP processing into Aβ₃₈; and (3) have increased selectivity for reduction of Aβ₄₂ relative to inhibition of COX-1, COX-2, or both COX-1 and COX-2 enzymatic activity. In addition, the invention provides methods and materials related to identifying agents that can increase the risk of developing AD, or hasten the progression of AD, in a mammal. The invention also provides compositions and kits that can be used to prevent, delay, or reverse the progression of AD.

In one embodiment, the invention provides a method of preventing, delaying, or reversing the progression of AD by administering an Aβ₄₂ lowering agent to a mammal under conditions in which levels of Aβ₄₂ are reduced, levels of Aβ₃₈ are increased, and levels of Aβ₄₀ are unchanged. The Aβ₄₂ lowering agent also can increase the levels of one or more of Aβ₃₄, Aβ₃₆, Aβ₃₇, and Aβ₃₉.

The Aβ₄₂ lowering agent can be an NSAID, an NSAID derivative, an NSAID analogue, or any compound that reduces levels of Aβ₄₂, increases levels of Aβ₃₈, and has no effects on levels of Aβ₄₀, (i.e., levels of Aβ₄₀ are neither increased nor decreased). The Aβ₄₂ lowering agent can be an aryl propionic acid derivative, an aryl acetic acid derivative, or an amino carboxylic acid derivative. More specifically, the Aβ₄₂ lowering agent can be a structural derivative of an NSAID such as flufenmic acid, meclofenamic acid, fenoprofen, carprofen, ibuprofen, ketoprofen, and flurbiprofen. The Aβ₄₂ lowering agent also can be a structural derivative of 5-nitro-2-(3-phenylpropylamino) benzoic acid). Typically, the Aβ₄₂ lowering agent either (1) lacks COX-1, COX-2, or both COX-1 and COX-2 inhibiting activity, or (2) has a greater potency for lowering Aβ₄₂ levels than for inhibiting COX-1, COX-2, or both COX-1 and COX-2 enzymatic activity. In one embodiment, the Aβ₄₂ lowering agent is R-flurbiprofen (also known as tarenflurbil).

Aβ₄₂ lowering agents of the invention can be used to treat AD in a mammal such as a human. The mammal may not be diagnosed with AD, or may not have a genetic predisposition for AD.

In another embodiment, the invention provides a method for developing an Aβ₄₂ lowering agent. The method involves generating derivatives of the NSAIDs meclofenamic acid or flufenamic acid by altering the position of the carboxylic acid group on the phenyl ring or altering the position or type of substituents on the phenyl ring opposite the carboxylic acid group. Derivatives also can be generated by altering the bond connecting the two phenyl rings, altering the carboxylic acid group to propionic acid or another substituent, or performing any combination of these alterations. The derivative is then tested to determine its ability to decrease Aβ₄₂ levels while increasing Aβ₃₈ levels.

In another embodiment, the invention provides a method for developing an Aβ₄₂ lowering agent. The method involves generating derivatives of the NSAIDs fenoprofen, flurbiprofen, or carprofen. Derivatives can be generated by altering the position of the propionic acid group on the phenyl ring, or altering the position or type of substituents on the phenyl ring opposite the propionic acid group. Derivatives also can be generated by altering the bond connecting the two phenyl rings, altering the acetic acid group to carboxylic acid or another substituent, or performing any combination of these alterations. The derivative is then tested to determine its ability to decrease Aβ₄₂ levels while increasing Aβ₃₈ levels.

In another embodiment, the invention provides a method for developing an Aβ₄₂ lowering agent. The method involves generating derivatives of indomethacin by altering the carboxylic acid group to another substituent, altering the indole nitrogen to another substituent, or performing any combination of these alterations. The derivative is then tested to determine its ability to decrease Aβ₄₂ levels while increasing Aβ₃₈ levels.

In another embodiment, the invention provides a method for developing an Aβ₄₂ lowering agent. The method involves generating derivatives of sulindac sulfide by altering the methylthiol group, the propionic acid group, or the fluoride moiety to another substituent, or performing any combination of these alterations. The derivative is then tested to determine its ability to decrease Aβ₄₂ levels while increasing Aβ₃₈ levels.

In another embodiment, the invention provides a method for identifying an Aβ₄₂ lowering agent useful for preventing, delaying, or reversing the progression of Alzheimer's disease. The method involves treating a biological composition that has APP and an APP processing activity with a candidate Aβ₄₂ lowering agent under conditions in which APP processing occurs. An Aβ₄₂ lowering agent, useful for preventing, delaying, or reversing the progression of Alzheimer's disease, is one that, when present, decreases the level of Aβ₄₂ in the biological composition.

In another embodiment, the invention provides a method for identifying an Aβ₄₂ lowering agent useful for preventing, delaying, or reversing the progression of Alzheimer's disease. The method involves treating a biological composition that has Aβ₄₂ and an Aβ₄₂ catabolic activity with a candidate Aβ₄₂ lowering agent under conditions in which Aβ₄₂ catabolism occurs. An Aβ₄₂ lowering agent, useful for preventing, delaying, or reversing the progression of Alzheimer's disease, is one that, when present, decreases the level of Aβ₄₂ in a biological composition.

In another embodiment, the invention provides a method for identifying an Aβ₄₂ lowering agent that has a greater potency for lowering Aβ₄₂ levels than for inhibiting COX-1, COX-2, or both COX-1 and COX-2 enzymatic activity. The method involves identifying Aβ₄₂ lowering agents by screening for those having the ability to lower the level of Aβ₄₂ in a biological composition. The IC50 of the Aβ₄₂ lowering agent for Aβ₄₂ lowering can be determined by performing dose response studies. The Aβ₄₂ lowering agent is examined for the ability to inhibit COX-1, COX-2, or both COX-1 and COX-2 using in vitro COX-1 and COX-2 inactivation assays. The IC50 for Aβ₄₂ lowering is compared to the IC50 for COX-1, COX-2, or both COX-1 and COX-2 inhibition. An Aβ₄₂ lowering agent that has an greater potency for lowering Aβ₄₂ levels than for inhibiting COX-1, COX-2, or both COX-1 and COX-2 activity is one that has an IC50 for Aβ₄₂ lowering greater than ten-fold the IC50 for COX-1, COX-2, or both COX-1 and COX-2 inhibition. The greater potency for lowering Aβ₄₂ levels than for inhibiting COX-1, COX-2, or both COX-1 and COX-2 activity can be confirmed by demonstrating that administration of the Aβ₄₂ lowering agent to an animal reduces Aβ₄₂ levels at doses that do not inhibit or only minimally inhibit COX-1, COX-2, or both COX-1 and COX-2 activity such that significant COX-related side-effects do not occur.

In another embodiment, the invention provides a method for identifying an agent that increases the risk of developing, or hastens progression of, AD in a patient. The method involves exposing a biological composition that has APP and an APP processing activity to a candidate agent under conditions in which APP processing occurs. The level of Aβ₄₂ in the biological composition exposed to the candidate agent is compared to the level of Aβ₄₂ in a biological composition not exposed to the candidate agent. The candidate agent is one that can increase the risk of developing, or hasten the progression of, AD if an increase in the level of Aβ₄₂ in the biological composition exposed to the agent is observed when compared with the level of Aβ₄₂ in the biological composition not exposed to the agent.

In another embodiment, the invention provides a method for identifying an agent that increases the risk of developing, or hastens progression of, AD in a patient. The method involves exposing a biological composition that has Aβ₄₂ and an Aβ₄₂ catabolic activity to a candidate agent under conditions in which Aβ₄₂ catabolism occurs. The level of Aβ₄₂ in the biological composition exposed to the candidate agent is compared to the level of Aβ₄₂ in a biological composition not exposed to the candidate agent. The candidate agent is one that can increase the risk of developing, or hasten the progression of, AD if an increase in the level of Aβ₄₂ in the biological composition exposed to the agent is observed when compared with the level of Aβ₄₂ in the biological composition not exposed to the agent.

In another embodiment, the invention provides a composition consisting of an Aβ₄₂ lowering agent and an antioxidant. The antioxidant can be, without limitation, vitamin E, vitamin C, curcumin, and Gingko biloba.

In another embodiment, the invention provides a composition consisting of an Aβ₄₂ lowering agent and a non-selective secretase inhibitor.

In another embodiment, the invention provides a composition consisting of an Aβ₄₂ lowering agent and an acetylcholinesterase inhibitor.

In another embodiment, the invention provides kits containing (1) an Aβ₄₂ lowering agent and an antioxidant; (2) an Aβ₄₂ lowering agent and a non-selective secretase inhibitor; or (3) an Aβ₄₂ lowering agent and an acetylcholinesterase inhibitor. Kits can include instructions that indicate dose regimens for the Aβ₄₂ lowering agent, the antioxidant, the secretase inhibitor, and/or the acetylcholinesterase inhibitor.

In another embodiment, the invention provides for the use of an Aβ₄₂ lowering agent in the manufacture of a medicament for the treatment of AD. When administered to a patient, the medicament containing the Aβ₄₂ lowering agent is effective for reducing Aβ₄₂ levels without affecting Aβ₄₀ levels. The medicament also can increase Aβ₃₈ levels, and may also increase Aβ₃₄, Aβ₃₆, Aβ₃₇, or Aβ₃₉ levels. The Aβ₄₂ lowering agent in the medicament can be an aryl propionic acid derivative, an aryl acetic acid derivative, or an amino carboxylic acid derivative. More specifically, the Aβ₄₂ lowering agent in the medicament can be a structural derivative of an NSAID selected from the group consisting of flufenmic acid, meclofenamic acid, fenoprofen, carprofen, ibuprofen, ketoprofen, and flurbiprofen. The Aβ₄₂ lowering agent also can be a structural derivative of 5-nitro-2-(3-phenylpropylamino)benzoic acid). The Aβ₄₂ lowering agent in the medicament can lack COX-1, COX-2, or both COX-1 and COX-2 inhibiting activity. The Aβ₄₂ lowering agent in the medicament can have a greater potency, in vivo, for lowering Aβ₄₂ levels than for inhibiting COX-1, COX-2, or both COX-1 and COX-2 activity. The medicament can be used to treat AD in a mammal such as a human. The medicament can be used in a mammal that has not been diagnosed with AD, or in a mammal that does not have a genetic predisposition for AD.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a bar graph summarizing Aβ₄₂/Aβ₄₀ ratios and total Aβ levels determined for CHO cells expressing APP751 and PS-1 mutant M146L that had been treated with DMSO or with various concentrations of sulindac sulfide.

FIG. 2 is a bar graph summarizing Aβ₄₂/Aβ₄₀ ratios and total Aβ levels determined for human neuroglioma cells (HS683) expressing APP695 that had been treated with DMSO or with various concentrations of sulindac sulfide.

FIG. 3 is a bar graph summarizing Aβ₄₂/Aβ₄₀ ratios and total Aβ levels determined for CHO cells expressing APP751 and PS-1 mutant M146L that had been treated with ethanol or with various concentrations of ibuprofen.

FIG. 4 is a bar graph summarizing Aβ₄₂/Aβ₄₀ ratios and total Aβ levels determined for CHO cells expressing APP751 and PS-1 mutant M146L that had been treated with DMSO or with various concentrations of indomethacin.

FIG. 5 is a bar graph summarizing Aβ₄₂/Aβ₄₀ ratios and total Aβ levels determined for CHO cells expressing APP751 that had been treated with DMSO or with various concentrations of naproxen.

FIG. 6 is a bar graph comparing Aβ₄₂/Aβ₄₀ ratios and total Aβ levels in CHO cells expressing APP751 that had been treated with ethyl acetate or various concentrations of celecoxib.

FIG. 7 is a bar graph summarizing Aβ₄₂/Aβ₄₀ ratios and total Aβ levels determined for primary fibroblasts (from COX-1/COX-2 double-knockout mice) expressing APP 695 that had been treated with DMSO or various concentrations of sulindac sulfide.

FIG. 8 is two representative mass spectra of Aβ species secreted by CHO cells expressing APP751 after treatment with DMSO or 100 μM sulindac sulfide.

FIG. 9 is a bar graph illustrating ratios of Aβ₁₋₄₂, Aβ₁₋₃₉, Aβ₁₋₃₈, and Aβ₁₋₃₇ to Aβ₁₋₄₀ in cells treated with DMSO or sulindac sulfide.

FIG. 10 is a scattergram of Aβ₄₀ and Aβ₄₂ levels in the brains of Tg2576 mice after short-term NSAID treatment.

FIG. 11 is a summary of the structures of indomethacin and meclofenamic acid, possible side chain modifications, and the effects of these modifications on COX-1 and COX-2 activities.

FIG. 12 is a compilation of the structures of newly synthesized biphenyl amines.

FIG. 13 is a time course of Aβ₄₂ reduction in CHO APP695NL,I,his cell cultures treated with meclofenamic acid.

FIG. 14 is a bar graph illustrating the effect of NSAIDs on Aβ42 production in H4 APP 695 wild type cells. Cells treated with the vehicle (DMSO) alone were used as controls.

FIG. 15 is a bar graph comparing the effect of the S and R enantiomers of flurbiprofen and the effect of a mixture of R+S enantiomers on brain Aβ₄₂ levels in Tg2576 mice. Data are expressed as a % of the control. Both the mixture of R+S enantiomers and R-flurbiprofen alone lower Aβ₄₂ levels in 3-month old Tg2576 mouse brains (p<0.01).

### DETAILED DESCRIPTION

The invention relates to the use of Aβ₄₂ lowering agents to prevent, delay, or reverse the progression of AD. The invention is based on the discovery that some but not all NSAIDs can reduce the level of the pathogenic Aβ₄₂ form, when used at a sufficiently high amount. Such NSAIDs are useful for treating AD. In particular, it has been now discovered that some NSAIDs can selectively reduce Aβ₄₂, without substantially affect Aβ₄₀, and increase Aβ species that are small than Aβ₄₀ (such as Aβ₃₈). Therefore, in preferred embodiments, the invention provides methods and materials related to identifying Aβ₄₂ lowering agents, including NSAIDs, NSAID derivatives, and NSAID analogues that (1) can reduce the level of Aβ₄₂ by reducing APP processing into Aβ₄₂ or by increasing Aβ₄₂ catabolism; (2) increase the level of Aβ₃₈ by increasing APP processing into Aβ₃₈; and (3) have increased selectivity for reduction of Aβ₄₂ relative to inhibition of COX-1, COX-2, or both COX-1 and COX-2. In addition, the invention provides methods and materials related to identifying agents that can increase the risk of, or hasten the progression of, AD in a mammal, by increasing the processing of APP into Aβ₄₂, or decreasing the catabolism of Aβ₄₂. The invention also provides compositions and kits that can be used to prevent, delay, or reverse the progression of AD.

1. Aβ₄₂ Lowering Agents

Aβ₄₂ lowering agents include, without limitation, NSAIDs, NSAID derivatives, and NSAID analogues. NSAIDs can be FDA-approved NSAIDs. NSAID derivatives are compounds generated by modifying functional groups of known NSAIDs. Once modified, derivatives may or may not have the anti-inflammatory properties of the parent NSAIDs. Structural analogues of NSAIDs are compounds that are structurally similar to NSAIDs. Analogues also may not have the anti-inflammatory properties of the corresponding structurally similar NSAIDs to which they resemble. Analogues can also be specific enantiomers of an NSAID drug that is racemic.

NSAIDs are non-steroidal anti-inflammatory drugs that are distinct from steroidal drugs with anti-inflammatory properties such as corticosteroids. NSAIDs, many of which are organic acids, typically have analgesic (pain-killing), anti-inflammatory, and antipyretic (fever-reducing) properties. Some examples of NSAIDs include salicylic acid (Aspirin), ibuprofen (Motrin, Advil), naproxen (Naprosyn), sulindac (Clinoril), diclofenac (Voltaren), piroxicam (Feldene), ketoprofen (Orudis), diflunisal (Dolobid), nabumetone (Relafen), etodolac (Lodine), oxaprozin (Daypro), Meclofenamic acid (Meclofen) and indomethacin (Indocin). NSAIDs can be grouped into classes, for example, amino aryl carboxylic acid derivatives (e.g., flufenamic acid, meclofenamic acid); aryl acetic acid derivatives (e.g., indomethacin, sulindac); and aryl propionic acid derivatives (fenoprofen, ibuprofen, carprofen and flurbiprofen).

Although NSAIDs have multiple cellular effects (see Cronstein et al. (1995) Annu Rev Pharmacol Toxicol 35:449-62; and Amin et al. (1999) Cell Mol Life Sci 56:305-12), many act through direct inhibition of COX enzymes. COX enzymes oxidize arachidonic acids from membrane bound phospholipids to prostaglandins (see Smith et al (2000) Ann Rev Biochem 69: 145-82). Inhibition of COX enzymes and therefore prostaglandin synthesis is believed to underlie the analgesic and anti-inflammatory properties of aspirin and NSAIDs (see Dubois et al. (1998) FASEB J 12: 1063-73). There are two isoforms of COX: COX-1 and COX-2. Although COX-1 and COX-2 catalyze the same reaction, they are derived from two different genes. COX-1 is traditionally viewed as a constitutive or housekeeping enzyme while COX-2 is viewed as an inducible enzyme that is expressed during inflammatory circumstances. COX products, primarily prostaglandin E2, modulate classical signs of inflammation. Another COX product is thromboxane A2 that promotes platelet aggregation and vasoconstriction. Although COX is expressed in neurons, its function in the central nervous system is unclear.

Another target of NSAIDs is the peroxisome proliferator-activator receptor (PPAR) family of nuclear hormone receptors. The PPAR family consists of at least three subtypes: PPARα, PPARδ, and PPARγ (see Corton et al. (2000) Annu Rev Pharmacol Toxicol 40:491-518). These receptors are thought to function as ligand-dependent activators of transcription. All three PPAR members are modulated by NSAIDs, although in different ways. For example, NSAIDs activate the activities of PPARδ and PPARγ but inhibit PPARγ activity (see He et al. (1999) Cell 99:335-45). It is known that PPARγ expression is increased in brains of AD individuals (Kitamura et al. (1999) Biochem Biophys Res Commun 254:582-6), and that PPARγ agonists block Aβ-stimulated secretion of proinflammatory products of microglia, including IL-1 and TNF-α (see Combs et al. (2000) J Neurosci 20:558-67). It has been suggested that the beneficial effects of NSAIDs in AD may be mediated via their activity on PPARγ rather than or in addition to COX inhibition (Combs et al. (2000) J Neurosci 20:558-67). It is not known, however, what downstream genes are activated by PPARs, or whether they are involved in Aβ production.

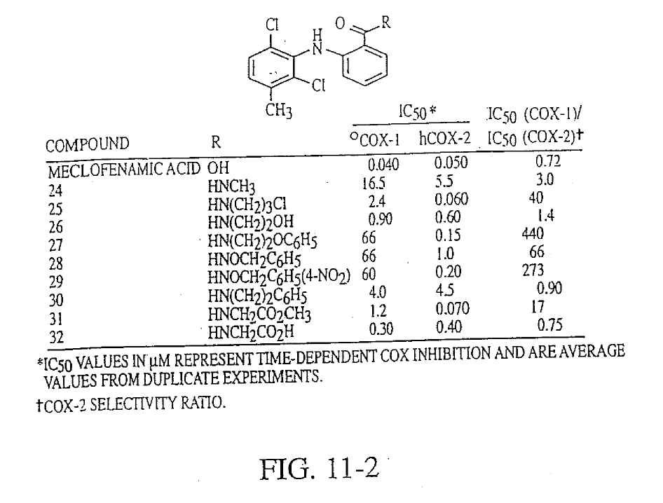

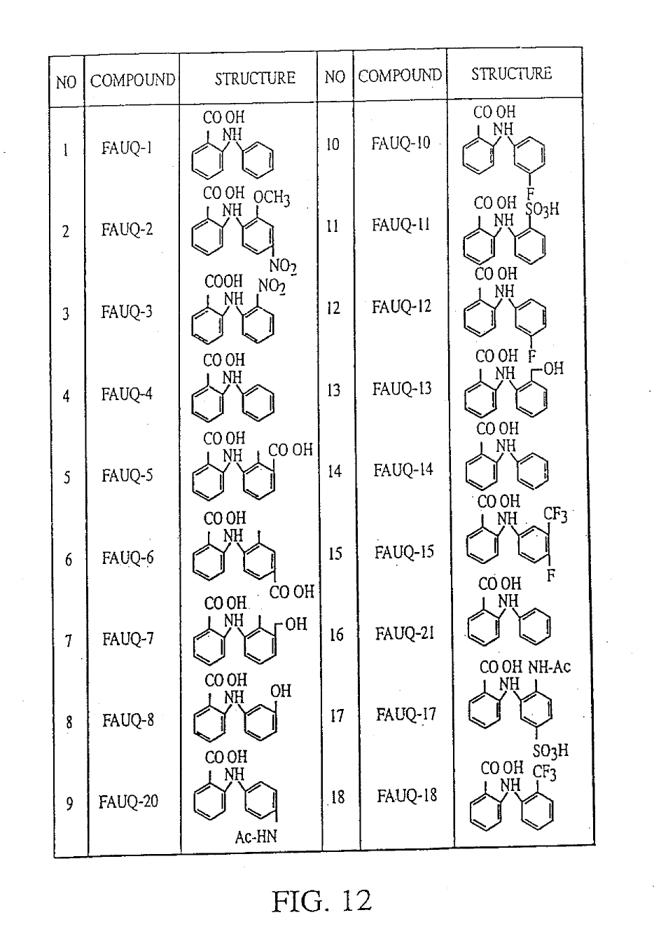

The term “Aβ₄₂ lowering agent” as used herein refers to an NSAID, an NSAID derivative, an NSAID analogue, or any compound that can reduce the level of Aβ₄₂ secreted from human cells. In addition, preferably the Aβ₄₂ lowering agents according to the present invention may also have one or both of the following two properties: (1) they do not substantially affect the level of Aβ₄₀ secretion from human cells; (2) they increase the level of Aβ₃₈ secreted from human cells. Such effects of Aβ₄₂ lowering agents can be determined in an assay provided in Examples 8 and 12 below. Examples of Aβ₄₂ lowering agents according to the present invention include, e.g., the NSAIDs, NSAID derivatives, and NSAID analogues disclosed in Examples 8, 9, 10, 15, 17 and 37, and particularly those in Tables 1 and 3 that meet the above definition for Aβ₄₂ lowering agents. Other specific examples of Aβ₄₂ lowering agents include the NSAID derivatives provided in FIGS. 11 and 12 that meet the above definition for Aβ₄₂ lowering agents. Generally examples of Aβ₄₂ lowering agents can be sulindac sulfide, flufenmic acid, mefenamic acid, fenoprofen, ibuprofen, indomethacin, and flurbiprofen, and derivatives or analogues of such NSAIDs that are aryl propionic acid, aryl acetic acid, or amino carboxylic acid. Methods of providing or identifying NSAIDs, NSAID derivatives and analogues that are Aβ₄₂ lowering agents are described in detail below.

In preferred embodiments, the Aβ₄₂ lowering agents according to the present invention can selectively reduce Aβ₄₂ production or secretion from human cells, and have all of the following three properties: (1) the ability to reduce the level of Aβ₄₂ either through reducing APP processing or increasing Aβ₄₂ catabolism, (2) substantially no effect on the level of Aβ₄₀, and (3) and the ability to increase the level of Aβ₃₈. In other words, in these preferred embodiments, the Aβ₄₂ lowering agents can reduce the ratio between the level of Aβ₄₂ and Aβ₄₀ (Aβ₄₂/Aβ₄₀) or between the level of Aβ₄₂ and the total level of Aβ peptides secreted human cells, and increase the ratio between the level of Aβ₃₈ and Aβ₄₀ (Aβ₄₂/Aβ₄₀) or between the level of Aβ₃₈ and the total level of Aβ peptides secreted human cells. The levels can be measured as levels of secretion of the individual or total Aβ peptides from human cells. The three properties are referred to collectively as the Alzheimer's-Aβ₄₂-NSAID (Aβ₄₂-NSAID) footprint. In addition to having the Aβ₄₂-NSAID footprint, an Aβ₄₂ lowering agent of the invention preferably can increase the level of secretion of Aβ forms smaller than Aβ₄₀ such as Aβ₃₄, Aβ₃₆, Aβ₃₇, and Aβ₃₉ from human cells.

As used herein, the terms “increase,” “decrease” and “reduce” refer to a change in any amount that is reproducible and significant. When characterizing an Aβ₄₂ lowering agent as one that “increases”, “decreases” or “reduces” an Aβ peptide, it is meant that the Aβ₄₂ lowering agent is such that in an assay in Example 8 or 12, the concentration of the Aβ₄₂ lowering agent required to achieve a 50% reduction or increase of the level of the secreted Aβ peptide is no greater than about 250 μM. The increase or decrease is comparative to a control in the absence of an Aβ₄₂ lowering agent as is clear from Example 8 or 12. A reproducible and significant change is differentiated from irreproducible or insignificant experimental variations in measurements by standard statistical analysis methods including analysis that involves comparison with changes observed for control agents known to have no effects on the levels of the Aβ forms of interest.

In some preferred embodiments, the Aβ₄₂ lowering agent lack COX-1, COX-2, or both COX-1 and COX-2 inhibiting activity. That is, at a concentration of about 250 μM, an Aβ₄₂ lowering agent does not have any detectable inhibitory activity against COX-1 or COX-2 enzyme, or exhibits less than 20% inhibition of COX-1 or COX-2 enzyme activity as determined using the method described in Kalgutkar et al. Proc. Nat'l. Acad. Sci. USA, 97:925-930 (2000). Specifically, in a time-dependent and competitive inhibition assays, recombinant human COX-2 (66 nM) or ovine COX-1 (44 nM) in 100 mM Tris-HCl buffer (pH 8.0) containing 500 μM phenol is incubated with a test compound in DMSO (0-66 μM) at 25° C. for 20 min and then analyzed for remaining COX activity by treatment with [1-¹⁴C]arachidonic acid (50 μM, 57 mCi/mmol) for 30 sec at 37° C. Competitive inhibition by the test compounds is studied by adding COX-1 (2 nM) or COX-2 (2 nM) to an incubation mixture containing [1-¹⁴C]arachidonate (2 μM) and inhibitor (0-20 μM). Isolation and quantification of radio-labeled prostanoid products is done by a known method. All IC₅₀ values should average determinations from two independent experiments.

2. Identification of Aβ₄₂ Lowering Agents Useful for Treating AD

Aβ₄₂ lowering agents can be identified from collections of NSAIDs, NSAID derivatives, NSAID analogues, or other compounds using the Aβ₄₂-NSAID footprint. Such compounds can be obtained from any commercial source. For example, NSAIDs, NSAID derivatives, and NSAID analogues can be obtained from Sigma, Biomol, Cayman Chemical, ICN, or from the web through the Chemnavigator website. Novel NSAIDs, novel NSAID derivatives, and novel NSAID analogues can be chemically synthesized using methods described in many published protocols. NSAIDs, NSAID derivatives, and NSAID analogues can be synthesized with altered potency for their known targets such as COX-1 and COX-2. For example Kalgutkar et al., Proc. Nat'l. Acad. Sci. USA, 97:925-930 (2000) have made derivatives of indomethacin and meclofenamic acid and Bayly et al, Biorg. Med. Chem. Letters 9:307-312 (1999) have made derivatives of Flurbiprofen. Indeed, because of the great effort to engineer NSAIDs so that they preferentially inhibit COX-2 rather than non-selectively inhibit COX-1 and COX-2, there have been numerous published articles and patents documenting synthesis derivatives of known NSAIDs. See Dewitt, Mol. Pharm., 55:625-631 (1999).

It is recognized that some NSAID derivatives or NSAID analogues generated can have (1) increased potency for lowering Aβ₄₂ levels and (2) decreased potency for COX inhibition. Although derivatives and analogues may no longer be considered NSAIDs since they may lack anti-inflammatory properties, Aβ₄₂ lowering agents can include such NSAID derivatives and NSAID analogues.

Aβ₄₂ lowering agents that have the Aβ₄₂-NSAID footprint can be identified using cell free assays, in vitro cell-based assays, and in vivo animal studies. Aβ₄₂ lowering agents can be dissolved in any suitable vehicle for in vitro cell culture studies or in vivo animal or human studies. A vehicle is an inert solvent in which a compound can be dissolved for administration. It is recognized that for any given Aβ₄₂ lowering agent, a vehicle suitable for in vitro cell culture studies or in vivo animal studies may not be the same as the vehicle used for human treatment. Some examples of suitable vehicles for cell culture or animal studies include water, dimethyl sulfoxide, ethanol, and ethyl acetate.

To identify Aβ₄₂ lowering agents that reduce APP processing, a biological composition having an APP processing activity (i.e. an activity that processes APP into various Aβ forms, one of which is Aβ₄₂), is incubated with APP under conditions in which APP processing occurs. To identify Aβ₄₂ lowering agents that increase Aβ₄₂ catabolism, a biological composition having Aβ₄₂ catabolic activity is incubated with Aβ₄₂ under conditions in which Aβ₄₂ catabolism occurs. Depending on the nature of the biological composition, the APP or Aβ₄₂ substrate can be added to the biological composition, or, each or both can be a component of the biological composition. APP processing or Aβ₄₂ catabolism is allowed to take place in the presence or absence of the candidate Aβ₄₂ lowering agent. The level of Aβ₄₂ generated from APP processing or the level of Aβ₄₂ remaining after the catabolic reaction, in the presence and absence of the candidate Aβ₄₂ lowering agent, is determined and compared. Aβ₄₂ lowering agents useful for treating AD are those that reduce the level of Aβ₄₂ either by reducing APP processing into Aβ₄₂ or by enhancing Aβ₄₂ catabolism and increasing Aβ₃₈ production.

The biological composition having an APP processing and/or catabolic activity can be a cell-free biological sample. For example, a cell-free biological sample can be a purified or partially purified enzyme preparation; it also can be a cell lysate generated from cells able to process APP into Aβ₄₂ or from cells able to catabolize Aβ₄₂. Cell lysates can be prepared using known methods such as, for example, sonication or detergent-based lysis. In the case of an enzyme preparation or cell lysate, APP can be added to the biological composition having the APP processing activity, or Aβ₄₂ can be added to the biological composition having Aβ₄₂ catabolic activity.

In addition, the biological composition can be any mammalian cell that has an APP processing activity as well as a nucleic acid vector encoding APP. Alternatively, the biological composition can be any mammalian cell that has Aβ catabolic activity as well as a nucleic acid vector or a viral nucleic acid-based vector containing a gene that encodes Aβ₄₂. The vector typically is an autonomously replicating molecule, a molecule that does not replicate but is transiently transfected into the mammalian cell, or a vector that is integrated into the genome of the cell. Typically, the mammalian cell is any cell that can be used for heterologous expression of the vector-encoded APP or Aβ₄₂ in tissue culture. For example, the mammalian cell can be a Chinese hamster ovary (CHO) cell, a fibroblast cell, or a human neuroglioma cell. The mammalian cell also can be one that naturally produces APP and processes it into Aβ₄₂, or one that naturally produces and catabolizes Aβ₄₂.

Further, the biological composition can be an animal such as a transgenic mouse that is engineered to over-express a form of APP that then is processed into Aβ₄₂. Alternatively, the animal can be a transgenic mouse that is engineered to over-express Aβ₄₂. Animals can be, for example, rodents such as mice, rats, hamsters, and gerbils. Animals also can be rabbits, dogs, cats, pigs, and non-human primates, for example, monkeys.

To perform an in vitro cell-free assay, a cell-free biological sample having an activity that can process APP into Aβ₄₂ is incubated with the substrate APP under conditions in which APP is processed into various Aβ forms including Aβ₄₂ (see Mclendon et al. (2000) FASEB 14:2383-2386). Alternatively, a cell-free biological sample having an activity that can catabolize Aβ₄₂ is incubated with the substrate Aβ₄₂ under conditions in which Aβ₄₂ is catabolized. To determine whether a candidate Aβ₄₂ lowering agent has an effect on the processing of APP into Aβ₄₂ or the catabolism of Aβ₄₂, two reactions are compared. In one reaction, the candidate Aβ₄₂ lowering agent is included in the processing or catabolic reaction, while in a second reaction, the candidate Aβ₄₂ lowering agent is not included in the processing or catabolic reaction. Levels of the different Aβ forms produced in the reaction containing the candidate Aβ₄₂ lowering agent are compared with levels of the different Aβ forms produced in the reaction that does not contain the candidate Aβ₄₂ lowering agent.

The different Aβ forms can be detected using any standard antibody based assays such as, for example, immunoprecipitation, western hybridization, and sandwich enzyme-linked immunosorbent assays (ELISA). Different Aβ forms also can be detected by mass spectrometry; see, for example, Wang et al. (1996) J Biol Chem 271:31894-902. Levels of Aβ species can be quantified using known methods. For example, internal standards can be used as well as calibration curves generated by performing the assay with known amounts of standards.

In vitro cell-based assays can be used determine whether a candidate Aβ₄₂ lowering agent has an effect on the processing of APP into Aβ₄₂ or an effect on catabolism of Aβ₄₂. Typically, cell cultures are treated with a candidate Aβ₄₂ lowering agent. Then the level of Aβ₄₂ in cultures treated with a candidate Aβ₄₂ lowering agent is compared with the level of Aβ₄₂ in untreated cultures. For example, mammalian cells expressing APP are incubated under conditions that allow for APP expression and processing as well as Aβ₄₂ secretion into the cell supernatant. The level of Aβ₄₂ in this culture is compared with the level of Aβ₄₂ in a similarly incubated culture that has been treated with the candidate Aβ₄₂ lowering agent. Alternatively, mammalian cells expressing Aβ₄₂ are incubated under conditions that allow for Aβ₄₂ catabolism. The level of Aβ₄₂ in this culture is compared with the level of Aβ₄₂ in a similar culture that has been treated with the candidate Aβ₄₂ lowering agent.

In vivo animal studies also can be used to identify Aβ₄₂ lowering agents useful for treating AD. Typically, animals are treated with a candidate Aβ₄₂ lowering agent and the levels of Aβ₄₂ in plasma, CSF, and/or brain are compared between treated animals and those untreated. The candidate Aβ₄₂ lowering agent can be administered to animals in various ways. For example, the candidate Aβ₄₂ lowering agent can be dissolved in a suitable vehicle and administered directly using a medicine dropper or by injection. The candidate Aβ₄₂ lowering agent also can be administered as a component of drinking water or feed. Levels of Aβ in plasma, cerebral spinal fluid (CSF), and brain are determined using known methods. For example, levels of Aβ₄₂ can be determined using sandwich ELISA or mass spectrometry in combination with internal standards or a calibration curve. Plasma and CSF can be obtained from an animal using standard methods. For example, plasma can be obtained from blood by centrifugation, CSF can be isolated using standard methods, and brain tissue can be obtained from sacrificed animals.

When present in an in vitro or in vivo APP processing or Aβ₄₂ catabolic reaction, Aβ₄₂ lowering agents reduce the level of Aβ₄₂ generated by APP processing or remaining following Aβ catabolism. For example, in an in vitro cell-free assay, the level of Aβ₄₂ is reduced due to either a reduction of APP processing or an increase in Aβ₄₂ catabolism in the presence the Aβ₄₂ lowering agent. In an in vitro cell culture study, a reduction in the level of Aβ₄₂ secreted into the supernatant results from the effect of the Aβ₄₂ lowering agent on either a reduction in processing of APP into Aβ₄₂ or an increased catabolism of Aβ₄₂. Similarly, in animal studies, a reduction in the level of Aβ₄₂ that can be detected in plasma, CSF, or brain is attributed to the effect of the Aβ₄₂ lowering agent on either a reduction in the processing of APP into Aβ₄₂ or an increase in the catabolism of Aβ₄₂.

The level of Aβ₄₂ can be reduced by a detectable amount. For example, treatment with an Aβ₄₂ lowering agent leads to a 0.5, 1, 3, 5, 7, 15, 20, 40, 50, or more than 50% reduction in the level of Aβ₄₂ generated by APP processing or remaining following Aβ₄₂ catabolism when compared with that in the absence of the Aβ₄₂ lowering agent. Preferably, treatment with the Aβ₄₂ lowering agent leads to at least a 20% reduction in the level of Aβ₄₂ generated when compared to that in the absence of Aβ₄₂ lowering agent. More preferably, treatment with an Aβ₄₂ lowering agent leads to at least a 40% reduction the level of Aβ₄₂ when compared to that in the absence of an Aβ₄₂ lowering agent.

Typically, the Aβ₄₂ lowering agent-associated reduction of Aβ₄₂ levels is accompanied by an increase in the level of Aβ₃₈. In contrast, (1) the level of Aβ₄₀ generated by APP processing or Aβ₄₂ catabolism in cell-free assays, (2) the level of Aβ₄₀ secretion into culture supernatants in cell-based assays, or (3) the level of Aβ₄₀ detected in blood plasma, CSF, or brains of animals treated with Aβ₄₂ lowering agent, is substantially unchanged or not affected.

Aβ₄₂ lowering agents of the invention may lack COX inhibitory activity or have reduced COX-1, COX-2, or both COX-1 and COX-2 activity. COX inhibitory activity can be determined using known methods. For example, COX inhibitory activity can be determined using the method described in Kalgutkar et al. (2000) PNAS 97:925-930.

A method to identify NSAID derivatives and NSAID analogues that possess Aβ₄₂ lowering ability and have altered COX activity is described. NSAID derivatives and NSAID analogues of aminocarboxylic acids, arylacetic acids and arylpoprionic acids can be tested for their ability to lower Aβ₄₂ and increase Aβ₃₈ in cultured cells and in animals (as described herein). They also can be tested simultaneously for their ability to inhibit COX-1 and COX-2 using in vitro assays as described by Kalgutkar et al. (2000) PNAS 97:925-930.

Derivatives of the NSAIDs sulindac, meclofenamic acid, flufenamic acid, indomethacin, carprofen, fenoprofen, and flurbiprofen that can be tested include the following:

(1) meclofenamic acid and flufenamic acid derivatives in which (a) the position of the carboxylic acid substituent on the phenyl ring is altered to a different position on the same phenyl ring, (b) the position(s) of the substituent(s) on the phenyl ring opposite the carboxylic acid substituent are altered to different position(s) on the same phenyl ring, (c) the substituent(s) on the phenyl ring opposite the carboxylic acid substituent are altered, e.g., to halogen, C₁₋₃ alkyl, C₁₋₃ haloalkyl, hydroxyl, nitro, etc., and optionally the same phenyl ring is substituted with one or more additional substituents such as halogen, C₁₋₃ alkyl, C₁₋₃ haloalkyl, hydroxyl, etc., (d) the bond connecting the two phenyl rings is altered, e.g., by changing —NH— to —(CH₂)₀₋₃—NH—(CH₂)₀₋₃, (e) the carboxylic acid substituent is altered to, e.g., ester, sulfonate, amide, sulfonamide, or another carboxylic acid or carboxylic acid derivative, such as acetic acid or propionic acid or other derivatives, e.g., —CH(CH₂CH₃)C(═O)OH, —C(CH₃)(CH₂CH₃)C(═O)OH, —CH═C(CH₃)C(═O)OH, —C(CH₂CH₃)₂C(═O)OH—CH₂OH, —C(═O)H, —CH(OH)OCH₃, —CH(CH₃)tetrazole, —CH₂C(═O)OH, —C(CH₃)₂C(═O)OH, and —NHC(═O)CH₃, or (e) any combination of these alterations;

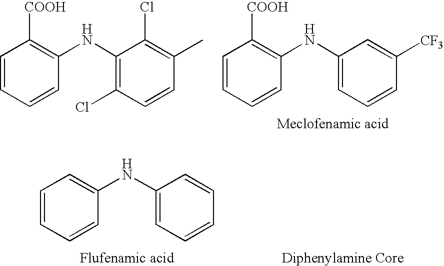

Several exemplary derivatives and analogues of Meclofenamic acid or Flufenamic acid suitable as Aβ₄₂ lowering agents according to the present invention are: 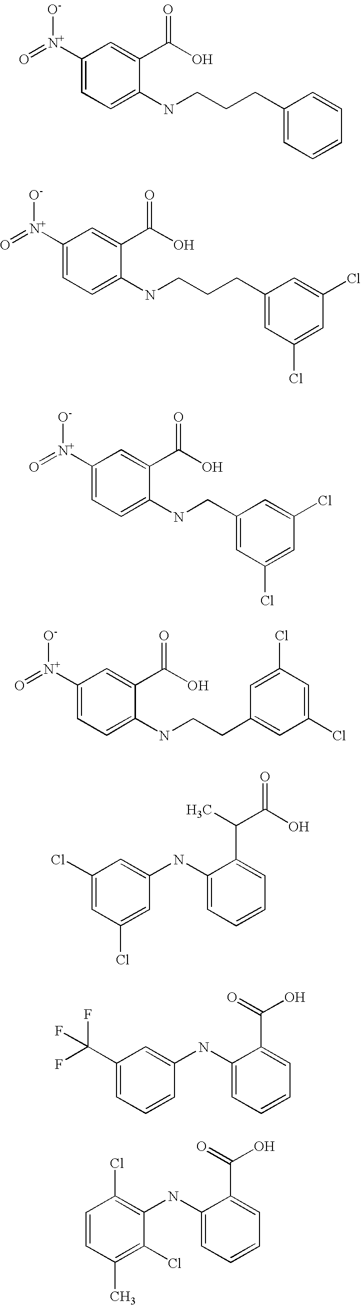 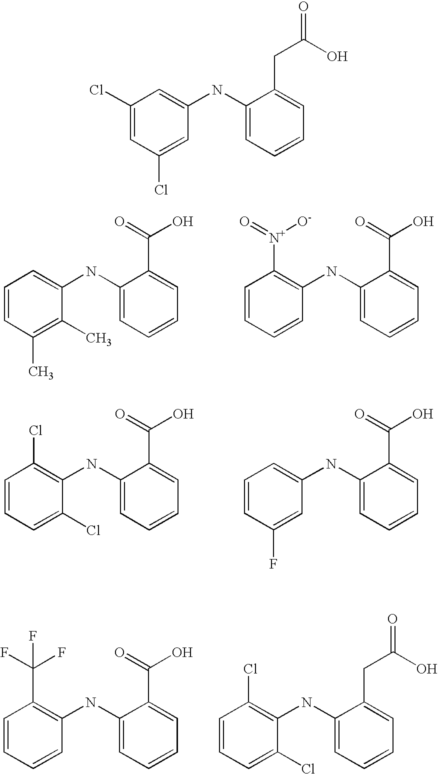
Additional compounds in this class are shown in FIG. 12.

(2) flurbiprofen derivatives in which (a) the position of the propionic acid substituent on the phenyl ring is altered to a different position in the same phenyl ring, (b) the position of fluoride on the phenyl ring is altered to a different position, and optionally the same phenyl ring is substituted with one or more additional substituents such as halogen, C₁₋₃ alkyl, C₁₋₃ haloalkyl, hydroxyl, etc., (c) the fluoride on the phenyl ring proximal to the propionic acid substituent is altered, e.g., to one or more substituents such as Cl, Br, I, C₁₋₃ alkyl, C₁₋₃ haloalkyl, hydroxyl, etc., and optionally the same phenyl ring is substituted with one or more additional substituents such as halogen, C₁₋₃ alkyl, C₁₋₃ haloalkyl, hydroxyl, etc., (c) the phenyl ring distal to the propionic acid substituent is substituted with, e.g., one or more subsitituents such as halogen, C₁₋₃ alkyl, C₁₋₃ haloalkyl, hydroxyl, or combinations thereof, (d) the bond connecting the two phenyl rings is altered, e.g., to a —(CH₂)₁₋₃, —NH—, etc., (e) the propionic acid substituent is altered to e.g., ester, sulfone, amide, sulfonamide, or another carboxylic acid derivative, such as acetic acid (—CH₂C(═O)OH), —C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH—CH₂OH, —C(═O)H, —CH(OH)OCH₃, —CH(CH₃)tetrazole, —CH₂C(═O)OH, —C(CH₃)₂C(═O)OH, and —NHC(═O)CH₃, or (f) any combination of these alterations.

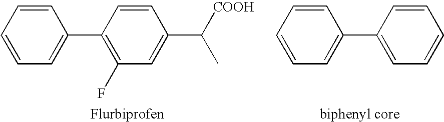

Examples of such derivatives include those disclosed in e.g., U.S. Pat. Nos. 1,091,403, 1,116,432, 1,359,987, 1,382,996, 1,396,726, 4,367,238, 4,699,925, 5,994,379; Japan Patent No. 08099924A2, the compounds and synthesis thereof in these patents being incorporated by references.

Several exemplary derivatives and analogues of flurbiprofen suitable as Aβ₄₂ lowering agents according to the present invention are:

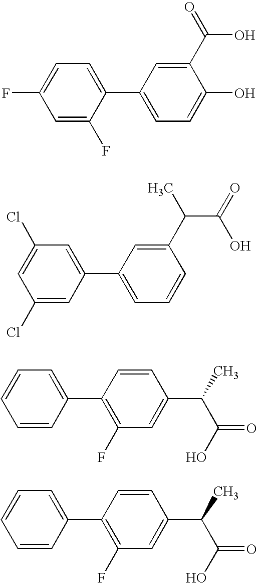

(2) fenoprofen derivatives in which (a) the position of the propionic acid substituent on the phenyl ring is altered to a different position on the same phenyl ring, (b) the phenyl ring opposite the propionic acid substituent is substituted with, e.g., one or more substituents such as halo, C₁₋₃ alkyl, C₁₋₃ alkoxy, C₁₋₃ haloalkyl, C₁₋₃ haloalkoxy, hydroxyl, (c) the phenyl ring proximate to the propionic acid group is additionally substituted with one to four substituents such as halo, C₁₋₃ alkyl, C₁₋₃ alkoxy, C₁₋₃ haloalkyl, C₁₋₃ haloalkoxy, hydroxyl, etc., (d) the propionic acid substituent is altered to e.g., ester, sulfonate, amide, sulfonamide, or another carboxylic acid or derivative thereof, e.g., acetic acid (—CH₂C(═O)OH), —C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH—CH₂OH, —C(═O)H, —CH(OH)OCH₃ , —CH(CH₃)tetrazole, —CH₂C(═O)OH, —C(CH₃)₂C(═O)OH, and —NHC(═O)CH₃, or (e) any combination of these alterations.

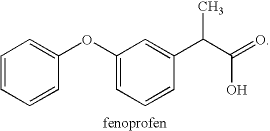

Several exemplary derivatives and analogues of fenoprofen suitable as Aβ₄₂ lowering agents according to the present invention are:

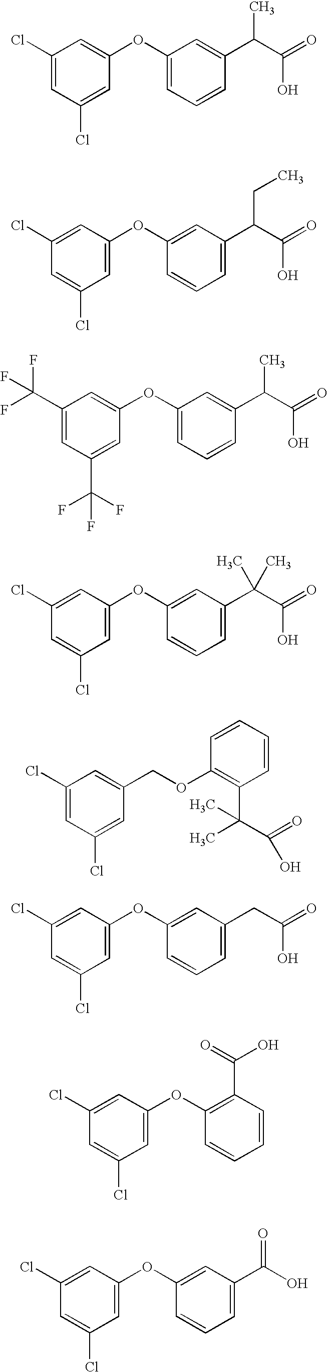

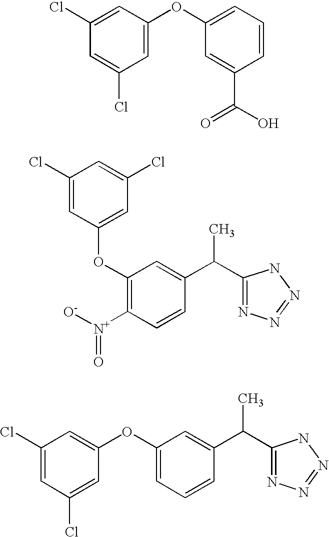

(3) indomethacin derivatives in which (a) the carboxylic acid group on indomethacin is altered to another substituent, e.g., ester, sulfonate, amide, sulfonamide, or another carboxylic acid or derivative thereof, e.g., such as —C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH—CH₂OH, —C(═O)H, —CH(OH)OCH₃, —CH(CH₃)tetrazole, —CH₂C(═O)OH, —C(CH₃)₂C(═O)OH, and —NHC(═O)CH₃, (b) the position of the Cl substituent is changed to another position on the same phenyl ring, (c) the Cl substituent is altered to a different substituent such as F, Br, hydroxyl, C₁₋₃ alkyl optionally substituted with 1-3 halo, and C₁₋₃ alkoxy optionally substituted with 1-3 halo, wherein the position can be at any one of the five positions on the same phenyl ring, and optionally the phenyl ring may be additionally substituted with one or more substituents such as halo, hydroxyl, C₁₋₃ alkyl, C₁₋₃ alkoxy, etc., (d) the 2-methyl on the indole core is altered to e.g., C₂₋₃ alkyl, (e) the methoxy group is changed to another position on the same phenyl ring, (f) the methoxy group is changed to a different subsitutuent, e.g., C₂₋₃ alkoxy optionally substituted with 1-3 halo, C₁₋₃ alkylthio optionally substituted with 1-3 halo, wherein the position can be at any one of the five positions on the phenyl ring, and optionally the phenyl ring may be additionally substituted with one or more substituents such as halo, hydroxyl, C₁₋₃ alkyl, C₁₋₃ alkoxy, etc., or (c) any combination of the two;

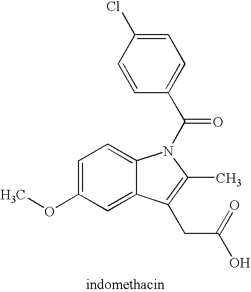

Examples of such derivatives include those disclosed in e.g., U.S. Pat. Nos. 3,161,654, 3,647,858, 3,654,349 and 5,436,265, the compounds and synthesis thereof in these patents being incorporated by references.

Several other exemplary derivatives and analogues of indomethacin suitable as Aβ₄₂ lowering agents according to the present invention are:

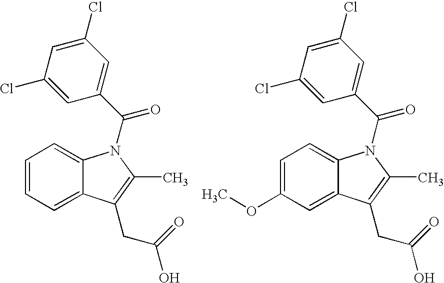

(4) sulindac sulfide in which (a) the methylthio derivative of sulindac sulfide is altered to other substituents, e.g., C₂₋₃ alkylthio optionally substituted with 1-3 halo, C₁₋₃ alkoxy optionally substituted with 1-3 halo, wherein the position can be at any one of the five positions on the phenyl ring, and optionally the phenyl ring may be additionally substituted with one or more substituents such as halo, hydroxyl, C₁₋₃ alkyl, C₁₋₃ alkoxy, etc., (b) the methylthio group can be altered to a different position on the same phenyl ring, (c) the propionic acid derivative is altered to another substituent, e.g., ester, sulfonate, amide, sulfonamide, or another carboxylic acid or derivative thereof, e.g., such as acetic acid (—CH₂C(═O)OH), —C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH—CH₂OH, —C(═O)H, —CH(OH)OCH₃ , —CH(CH₃)tetrazole, —CH₂C(═O)OH, —C(CH₃)₂C(═O)OH, and —NHC(═O)CH₃, (d) the position fluoride is altered to a different position at the same phenyl ring, (e) the fluoride is altered to a different substituent such as Cl, Br, hydroxyl, C₁₋₃ alkyl optionally substituted with 1-3 halo, and C₁₋₃ alkoxy optionally substituted with 1-3 halo, wherein the position can be at any one of the five positions on the same phenyl ring, and optionally the phenyl ring may be additionally substituted with one or more substituents such as halo, hydroxyl, C₁₋₃ alkyl, C₁₋₃ alkoxy, etc., or (d) any combination of the above.

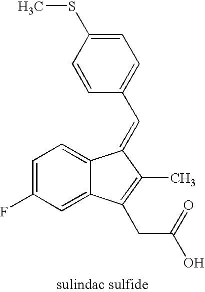

Examples of such derivatives include those disclosed in e.g., U.S. Pat. Nos. 3,161,654, 3,647,858, 3,654,349 and 5,436,265, the compounds and synthesis thereof in these patents being incorporated by references.

(5) ibuprofen derivatives in which (a) the position of the propionic acid substituent on the phenyl ring is altered to a different position in the same phenyl ring, (b) the position of the isobutyl group on the phenyl ring is altered to a different position, and optionally the same phenyl ring is substituted with one or more additional substituents such as halogen, C₁₋₃ alkyl, C₁₋₃ haloalkyl, hydroxyl, etc., (c) the propionic acid substituent is altered to e.g., ester, sulfone, amide, sulfonamide, or another carboxylic acid derivative, such as acetic acid (—CH₂C(═O)OH), —C(═O)OH, —CH(CH₂CH₃)C(═O)OH, —C(CH₃)(CH₂CH₃)C(═O)OH, —CH═C(CH₃)C(═O)OH, —C(CH₂CH₃)₂C(═O)OH —CH₂OH, —C(═O)H, —CH(OH)OCH₃, —CH(CH₃)tetrazole, —CH₂C(═O)OH, —C(CH₃)₂C(═O)OH, —NHC(═O)CH₃, cycloalkyl-(CH₂)₀₋₃COOH or ester thereof, (d) the isobutyl group on the phenyl ring is substituted by (altered to) another substituent such as ethyl, propyl, isopropyl, C₅₋₇ alkyl, C₁₋₆ haloalkyl, C₁₋₆ hydroxyalkyl, or (e) any combination of these alterations.

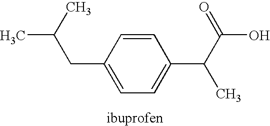

Examples of such ibuprofen derivatives include those disclosed in e.g., U.S. Pat. No. 3,746,751, the compounds and synthesis thereof being incorporated herein by reference.

In addition structural analogues of NSAIDs that possess Aβ₄₂ lowering ability, identified by pharamacophore searches (Perola et al., (2000) J. Med Chem. 43: 401-408) or other computer based structural comparison programs of commercially available compounds can be tested for Aβ₄₂ lowering activity, ability to increase Aβ₃₈, and COX inhibition as described herein.

Thus, the present invention provides methods of treating, or delaying the onset of, neurodegenerative disorders mediated by Aβ₄₂ including dementia, and particularly Alzheimer's disease or mild cognitive impairment, using one or more Aβ₄₂-lowering agents provided according to the present invention. The methods generally include the steps of identifying a patient in need of the treatment or delay; and administering to the patient an Aβ₄₂-reducing effective amount of an Aβ₄₂-lowering agent which can reduce the level of Aβ₄₂ secretion from a human cell. In some embodiments, the Aβ₄₂-lowering agent is characterized by the ability to reduce the level of Aβ₄₂ secretion without substantially affecting the level of Aβ₄₀ secretion. In specific embodiments, the Aβ₄₂-lowering agent is characterized by the ability to reduce the level of Aβ₄₂ secretion, increase the level of Aβ₃₈ secretion without substantially affecting the level of Aβ₄₀ secretion in assays performed in Examples 8 and 12. For example, the Aβ₄₂-lowering agent can be an NSAID, or a structural derivative or analogue thereof as described in detail above. Preferably, the Aβ₄₂-lowering agent reduces the plasma concentration of Aβ₄₂ in the patient treated. Preferably, the Aβ₄₂-lowering agent is a compound described above that causes at least 50% reduction in Aβ₄₂/Aβ₄₀ ratio at about 10 μM, 25 μM, 50 μM, 100 μM, 200 μM, or 250 μM in the assay of Example 8. Also preferably, the Aβ₄₂-lowering agent causes at least a two-fold increase in Aβ₃₈/Aβ₄₀ ratio at about 10 μM, 25 μM, 50 μM, 75 μM in the assay of Example 12.

The patients to be treated may have been diagnosed of mild cognitive impairment (MCI), mild Alzheimer's disease or mild to moderate disease. The patients may also be those who are free of any symptoms or signs of mild cognitive impairment or Alzheimer's disease, but desire to prevent or delay the onset of such symptoms or signs. Thus, the treatment can be applied to the patients at any progression stage of Alzheimer's disease, including prevention before the onset of mild cognitive impairment, or onset of Alzheimer's disease such that the onset is delayed, and treatment upon the detection of certain symptoms or signs of MCI or Alzheimer's disease to delay the onset of other symptoms or signs, slow the worsening of the symptoms or signs, or reverse the progression of the disease by improving the symptoms or signs.

In certain embodiments, either before or after the treatment with the Aβ₄₂-lowering agent or both, at least one of the following is monitored: (1) the plasma or CSF concentration of Aβ₄₂, (2) the plasma or CSF concentration of Aβ₄₀, (3) the plasma or CSF concentration of Aβ₃₄, Aβ₃₆, Aβ₃₇, or Aβ₃₉, (4) the plasma or CSF concentration of Aβ₃₈, (5) the Aβ₄₂/Aβ₄₀ concentration ratio in plasma or CSF, (6) the concentration ratio between Aβ₄₀ and one of Aβ₃₄, Aβ₃₆, Aβ₃₇, Aβ₃₈ and Aβ₃₉, or a combination thereof, plasma or CSF, (7) the ratio between the concentration of Aβ₄₂ and Aβ₃₈ in plasma or CSF, and (8) the total concentration of Aβ peptides in plasma or CSF.

The present invention also provides method for treating, or delaying the onset of, Alzheimer's disease, comprising (1) identifying a patient in need of the treatment or delay; (2) selecting an Aβ₄₂-lowering agent which reduces the level of Aβ₄₂ secretion, increases the level of Aβ₃₈ secretion without substantially affecting the level of Aβ₄₀ secretion from a human cell; and (3) administering to the patient an Aβ₄₂-reducing effective amount of said Aβ₄₂-lowering agent.

In preferred embodiments, the Aβ₄₂-lowering agent used in the various treatment methods of the present invention is an Aβ₄₂-reducing effective amount of (−)-(2R)-2-(2-fluorobiphenyl-4-yl)propanoic acid (tarenflurbil) or a pharmaceutically acceptable salt or ester thereof.

3. Identification of Mammals in Need of Treatment with an Aβ₄₂ Lowering Agent

Clinical symptoms of AD include, for example, progressive disorientation, memory loss, and aphasia; eventually, disablement, muteness, and immobility occur. Pathological indicators of AD include, for example, the presence of neurofibrillary tangles, neuritic plaques, and amyloid angiopathy. Preventing the progression of AD can be interpreted to mean preventing the onset or further development of clinical symptoms and/or pathological indicators of AD. For example, an individual who does not have clinical symptoms or pathological indicators of AD can be prevented from developing clinical symptoms or pathological indicators. Further, an individual who has a mild form of AD can be prevented from developing a more severe form of AD. Delaying the progression of AD can be interpreted to mean delaying the time of onset of AD-related symptoms and/or pathological indicators or slowing the rate of progression of AD, determined by the rate of development of clinical symptoms and pathological indicators. Reversing the progression of AD can be interpreted to mean that the severity of an AD condition has been lessened, i.e., the AD condition of an individual has changed from severe to less severe as indicated by fewer clinical symptoms or pathological indicators.

An individual can choose to take an Aβ₄₂ lowering agent as a preventative measure to avoid developing AD. For example, an individual with a genetic predisposition to AD can take an Aβ₄₂ lowering agent to prevent or delay the development of AD. A genetic predisposition can be determined based on known methods. For example, an individual can be considered to have a genetic predisposition to AD if the individual has a family history of AD. Genetic predisposition to AD also can include point mutations in certain genes such as the APP gene, the presenilin-1 or presenilin-2 gene, or the apolipoprotein E gene. Such mutations can predispose individuals to early-onset familial AD (FAD), increased risk of developing AD, or decreased age at onset of AD. (See page 1332, Table 30-2 of Cotran et al. (1999) Robbins Pathologic Basis of Disease, Sixth Edition, W.B. Saunders Company; and U.S. Pat. No. 5,455,169.) Furthermore, an individual who has clinical symptoms of, or has been diagnosed with, AD can take an Aβ₄₂ lowering agent to prevent or delay further progression of AD as well as to reverse the pathological condition of the disease.

An AD diagnosis can be made using any known method. Typically, AD is diagnosed using a combination of clinical and pathological assessments. For example, progression or severity of AD can be determined using Mini Mental State Examination (MMSE) as described by Mohs et al. (1996) Int Psychogeriatr 8:195-203; Alzheimer's Disease Assessment Scale-cognitive component (ADAS-cog) as described by Galasko et al. (1997) Alzheimer Dis Assoc Disord, 11 suppl 2:S33-9; the Alzheimer's Disease Cooperative Study Activities of Daily Living scale (ADCS-ADL) as described by McKhann et al. (1984) Neurology 34:939-944; and the NINCDS-ADRDA criteria as described by Folstein et al. (1975) J Psychiatr Res 12:189-198. In addition, methods that allow for evaluating different regions of the brain and estimating plaque and tangle frequencies can be used. These methods are described by Braak et al. (1991) Acta Neuropathol 82:239-259; Khachaturian (1985) Arch Neuro 42:1097-1105; Mirra et al. (1991) Neurology 41:479-486; and Mirra et al. (1993) Arch Pathol Lab Med 117:132-144.

4. Treatment of Mammals with Aβ₄₂ Lowering Agents

Aβ₄₂ lowering agents can be administered in any standard form using any standard method. For example, Aβ₄₂ lowering agents can be in the form of tablets or capsules that are taken orally. Aβ₄₂ lowering agents also can be in a liquid form that can be taken orally or by injection. Aβ₄₂ lowering agents also can be in the form of suppositories. Further, Aβ₄₂ lowering agents can be in the form of creams, gels, and foams that can be applied to the skin, or in the form of an inhalant.

Aβ₄₂ lowering agents can be administered at any dose that is sufficient to reduce levels of Aβ₄₂ in the blood plasma, CSF, or brain. Lower doses can be taken over a period of years to prevent and/or delay the progression of AD. Higher doses can be taken to reverse the progression of AD. Depending on the effectiveness and toxicity of a particular Aβ₄₂ lowering agent, an Aβ₄₂ lowering agent can be used at a dose of 0.1-50 mg/kg/day.

5. Compositions and Kits

The invention also provides pharmaceutical compositions containing combinations of an Aβ₄₂ lowering agent and an antioxidant effective in preventing, delaying, or reversing the progression of Alzheimer's disease. An Aβ₄₂ lowering agent of the invention that has the ability to reduce Aβ₄₂ levels can be combined with any antioxidant. The antioxidant can be a vitamin, for example vitamin E, vitamin C or curcumin; the antioxidant also can be Gingko biloba. The Aβ₄₂ lowering agent of the invention (e.g., tarenflurbil) that has the ability to reduce Aβ₄₂ levels and the antioxidant can be administered to a patient together or separately, in the method of the present invention, i.e., treating or delaying the onset of Alzheimer's disease or MCI. Other pharmaceutical compositions can include an Aβ₄₂ lowering agent and a non-selective secretase inhibitor or an acetylcholinesterase inhibitor. The Aβ₄₂ lowering agent (e.g., tarenflurbil) of the invention that has the ability to reduce Aβ₄₂ levels and the secretase inhibitor and/or acetylcholinesterase inhibitor can be administered to a patient together or separately, in the method of the present invention, i.e., treating or delaying the onset of Alzheimer's disease or MCI.

The pharmaceutical composition can be in any form, for example tablets, capsules, liquids, creams, gels, or suppositories and can include a suitable pharmaceutical carrier. In addition, the invention provides kits containing pharmaceutical compositions of Aβ₄₂ lowering agents and antioxidants as well as instructions that indicate dose regimens for effective use.

The invention will be further described in the following examples, which do not limit the scope of the invention described in the claims.

### EXAMPLES

### Example 1

### Cell Cultures, Drug Treatments, and Cell Toxicity Analysis

Cell cultures were maintained in standard cell culture media supplemented with 10% fetal bovine serum and 100 U/mL penicillin/streptomycin (Life Technologies Inc., Germany). Cell cultures consisted of the following: Chinese hamster ovary (CHO) cells that expressed human APP751 from a vector containing a gene encoding APP75 1; CHO cells that expressed both human APP751 and human mutant PS-1 (M146L) from vectors containing genes encoding APP751 and mutant PS-1 (M146L); CHO cells that expressed human mutant APP751 (V717F) from a vector containing a gene encoding mutant APP751 (V717F); human neuroglioma cells HS683 that expressed human wild type APP695 from a vector containing a gene encoding wild type APP695; HEK 293 cells that expressed human wild type APP695 from a vector containing a gene encoding wild type APP695; and embryonic fibroblasts (that had immortalized spontaneously) from COX-1 and COX-2 double-knockout mice.

The NSAIDs, sulindac sulfide (50 mM, Biomol, PA, USA), sulindac sulfone (50 mM, Biomol, PA, USA), naproxen (100 mM, Cayman Chemical, MI, USA), and aspirin (2.5 M, ICN Biomedicals, CA, USA) were dissolved in the vehicle DMSO. Indomethacin (50 mM, Biomol, PA, USA) and (S)-ibuprofen (250 mM, Biomol, PA, USA) were dissolved in ethanol. Celecoxib and rofecoxib capsules were obtained from and dissolved in ethyl acetate. For analyses of Aβ secretion, APP processing, and notch cleavage, cells were cultured in serum-containing media and pretreated overnight with a specific NSAID. The next day, media were changed and cultures were treated with the same NSAID for another 24 hours.

NSAID toxicity in CHO or HS683 cells was examined using standard MTT-assay (3-(4,5-Dimethyl-2-thiazolylyl)-2,5-diphenyl-2H-tetrazolium Bromide) or [³H]-thymidine incorporation assay. For cell toxicity studies, cells were treated with sulindac sulfide at concentrations up to 100 μM, indomethacin at concentrations up to 200 μM, and ibuprofen at concentrations up to 1 mM.

### Example 2

### Antibodies

Antibodies used included the following: 5A3 and 1G7, two monoclonal antibodies that recognized non-overlapping epitopes between residues 380-665 of APP; CT15, a polyclonal antibody that recognized the C-terminal fifteen amino acid residues of APP; 26D6, a monoclonal antibody that recognized amino acid residues 1-12 of the Aβ sequence; 9E10, a monoclonal antibody that recognized the myc-epitope sequence; anti-COX-2 antibody, a monoclonal antibody that recognized COX-2; and M-20, a polyclonal antibody that recognized COX-1. The antibodies 5A3, 1G7, CT15, and 26D6 were described by Koo et al. (1996) J Cell Sci 109:991-8; Sisodia et al. (1993) J Neurosci 13:3136-42; and Lu et al. (2000) Nat Med 6:397-404. The monoclonal antibody 9E10 was purchased from Calbiochem-Novobiochem, CA, USA. The monoclonal anti-COX-2 antibody was purchased from BD Transduction Laboratories, CA, USA. The polyclonal antibody M-20 was purchased from Santa Cruz Biotechnology, CA, USA.

### Example 3

### ELISA

Aβ was detected by sandwich enzyme-linked immunosorbent assay (ELISA) as described by Murphy et al. (2000) J Biol Chem 275:26277-84. Following NSAID treatment, culture supernatants were collected, and cell debris was removed by centrifugation. Complete protease inhibitor cocktail (Roche Molecular Biochemicals, IN, USA) was added to the media and Aβ₄₀ and Aβ₄₂ levels were quantified using end-specific Aβ ELISAs. All measurements were performed in duplicate.

### Example 4

### Adenoviral Infection of Embryonic Fibroblasts Derived from COX-1/COX-2 Double-Knockout Mice

The adenoviral vector containing a gene encoding APP695 was described by Yuan et al (1999) J Neurosci Methods 88:45-54. Primary fibroblasts derived from COX-1/COX-2 double-knockout mice were infected with 100 plaque-forming units (PFU) of viral vector per cell. Infection was performed in serum-free medium for two hours. Medium was changed and cells were treated with NSAIDs as described in Example 1.

### Example 5

### Analyses of APP and Notch Processing

Expression of holo-APP and APP C-terminal fragments (CTFs) was examined by Western blot analysis using antibody CT-15. APP secretion was examined by Western blotting using a mixture of 5A3/IG7 antibodies. APP turnover was examined by pulse labeling of CHO cells with 150 μCi [³⁵S]-methionine for fifteen minutes followed by a cold chase step for up to four hours. Cell lysates were immunoprecipitated with antibody CT-15, subjected to SDS-PAGE, and analyzed by phosphor imaging.

APP surface expression and internalization were measured as described by Koo et al. (1996) J Cell Sci 109:991-8. Iodinated antibody 1G7, at approximately 3-6 μCi/μg, was applied to confluent layers of CHO cells in binding medium (DMEM, 0.2% BSA, 20 mM HEPES [pH 7.4]) and incubated at 37° C. for thirty minutes. After incubation, cells were rapidly chilled on ice and the reaction was quenched by the addition of ice-cold binding medium. To remove unbound antibody, chilled cells were washed multiple times with ice-cold Dulbecco's phosphate-buffered saline (Life Technologies Inc.). Antibody bound to cell surface APP was detached by washing with ice-cold PBS (pH 2) for five minutes; this constituted the acid-labile APP antibody pool. Cells were lysed in 0.2 M NaOH; lysates contained the acid-resistant APP antibody pool. Acid-labile and acid-resistant APP antibody counts were measured by γ counting. The ratio of acid-resistant to acid-labile count was a measure of the internalized to the cell surface APP pool.

Two Notch-encoding vector constructs were used in examining Notch processing. These were a construct expressing a myc-tagged NH₂-terminal truncated Notch-1 polypeptide (NotchΔEMV), and a construct expressing only the Notch intracellular cytoplasmic domain (NICD) (see Kopan et al. (1996) Proc Natl Acad Sci USA 93:1683-8). In the construct expressing a myc-tagged NH₂-terminal truncated Notch-1 polypeptide, the start codon, a methionine at position 1726, was mutated to a valine to eliminate translation initiation.

### Example 6

### Mass Spectrometry

Secretion of Aβ peptides was analyzed using immunoprecipitation/mass spectrometry as described by Wang et al. (1996) J Biol Chem 271:31894-902. Briefly, 1 mL amount of culture supernatant was subjected to immunoprecipitation using the monoclonal antibody 4G8 (Senetek, CA, USA). Molecular masses and concentrations of Aβ peptides were measured using a matrix assisted laser desorption/ionization time-of-flight (MALDI-TOF) mass spectrometer. To compare the concentrations of individual Aβ species in culture supernatants, synthetic Aβ₁₂₋₂₈ peptides (Sigma, MO, USA) were added to the supernatant samples as internal standards and relative peak heights were calculated.

### Example 7

### Bicine/Urea Aβ Western Blot Analysis

Bicine/Urea Aβ western blot analysis was performed as described by Wiltfang et al. (1997) Electrophoresis 18:527-32. A 1 mL amount of culture supernatant was subjected to immunoprecipitation using monoclonal antibody 26D6. Immunoprecipitates were mixed with sample buffer and heated to 95 ° C. for five minutes. Eluant samples were separated on Bicine/Urea gels, then transferred to nitrocellulose membranes, and probed with antibody 26D6. Standard Aβ₁₋₄₀, Aβ₁₋₄₂ and Aβ₁₋₃₈ peptides (Sigma, MO, USA) were used for identification of the Aβ species.

### Example 8

### Cells Treated with the Non-Selective COX-Inhibitor Sulindac Sulfide Showed Reductions in Levels of Aβ42 Secretions

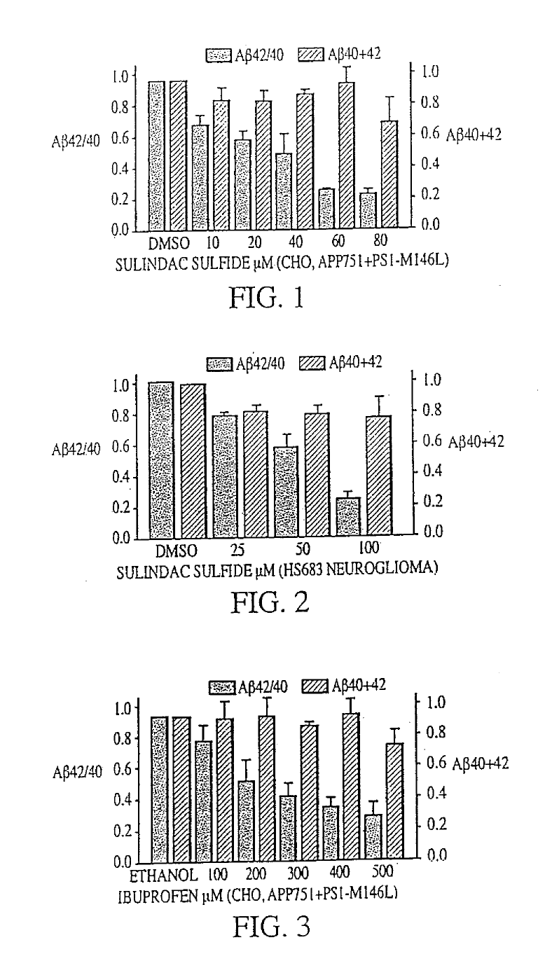

Cell cultures were treated with increasing concentrations of the NSAID sulindac sulfide. Levels of Aβ₄₀ and Aβ₄₂ in culture supernatants were analyzed using ELISA. FIG. 1 is a graph comparing the Aβ₄₂/Aβ₄₀ ratios of sulindac sulfide-treated CHO cell cultures expressing APP751 and the PS-1 mutant M146L. Aβ₄₂/Aβ₄₀ ratios and total Aβ levels (i.e., the sum of Aβ₄₀ and Aβ₄₂ values) were normalized to values obtained from DMSO-treated cells. Results shown were averages of two or three experiments performed in duplicate. CHO cell cultures treated with 40-60 μM sulindac sulfide showed a 50% reduction in Aβ₄₂/Aβ₄₀ ratios. No significant reduction in total Aβ level was observed. Therefore, treatment of CHO cells expressing APP and mutant PS-1 with the NSAID sulindac sulfide reduced the Aβ₄₂/Aβ₄₀ ratio by selectively reducing Aβ₄₂ secretion in a dose-dependent manner. This was confirmed in CHO cells that expressed wild type APP751 as well as those that expressed mutant APP V717F (data not shown). To rule out potential cell type-specific effects, Aβ secretion in response to sulindac sulfide treatment was examined in the human neuroglioma cell line HS683 that expressed APP695. FIG. 2 is a graph comparing Aβ₄₂/Aβ₄₀ ratios in HS683 cells expressing APP695 that were treated with DMSO with those of cells treated with various concentrations of sulindac sulfide. A dose-dependent reduction of Aβ₄₂ secretion, similar to that exhibited by CHO cells, was observed. Sulindac sulfide also reduced Aβ₄₂ secretion in kidney HEK293 cells and primary mouse embryonic fibroblasts (data not shown). No cell toxicity was observed at sulindac sulfide concentrations up to 100 μM (data not shown).

### Example 9

### Cells Treated with Other Non-Selective COX-Inhibitors Such as Ibuprofen and Indomethacin Showed Reductions in Levels of Aβ₄₂ Secretion

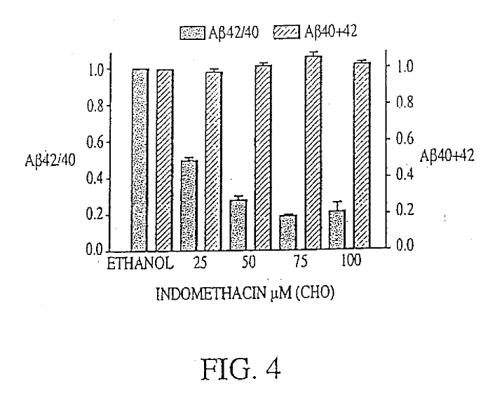

Cell cultures were treated with increasing concentrations of the NSAIDs ibuprofen and indomethacin. Aβ₄₀ and Aβ₄₂ levels in culture supernatants were analyzed using ELISA as described in Example 3. FIGS. 3 and 4 are graphs comparing Aβ₄₂/Aβ₄₀ ratios observed for CHO cells expressing APP751 and the PS-1 mutant M146L when treated with various concentrations of ibuprofen and indomethacin, respectively. Aβ₄₂/Aβ₄₀ ratios and total Aβ levels were normalized to values obtained from ethanol-treated cells. Results shown were averages of two or three experiments, each performed in duplicate. Dose dependent reductions in Aβ₄₂/Aβ₄₀ ratios by selective reductions of Aβ₄₂ secretion were observed for both ibuprofen and indomethacin. A 50% reduction in the Aβ₄₂/Aβ₄₀ ratio was reached at ibuprofen concentrations between 200-300 μM and at indomethacin concentrations between 25-50 μM. Total Aβ levels were not significantly affected at ibuprofen concentrations up to 500 μM (see FIG. 3) and at indomethacin concentrations up to 100 μM (see FIG. 4). No cell toxicity was observed in CHO cells treated with ibuprofen concentrations up to 1 mM or indomethacin concentrations up to 200 μM (data not shown).

### Example 10

### Reduction of Aβ42 Secretion is Not Associated with COX-Inhibitory Activity or With All NSAIDs

The effect of sulindac sulfone on Aβ42 secretion was examined. Sulindac sulfone is an oxidation product of the pro-drug sulindac. Like sulindac sulfide, sulindac sulfone inhibits proliferation and induces apoptosis in human cancer cell lines in vitro (see Piazza et al. (1995) Cancer Res 55:3110-6). In contrast to sulindac sulfide, sulindac sulfone is devoid of any inhibitory effect on COX. Cell cultures were treated with increasing concentrations of sulindac sulfone. Aβ₄₀ and Aβ₄₂ levels in culture supernatants were analyzed using ELISA. When CHO cells expressing APP 751 were treated with sulindac sulfone, no changes in Aβ₄₂/Aβ₄₀ ratios were observed with sulindac sulfone concentrations of up to 400 μM (data not shown). The inability to reduce Aβ₄₂ secretion by the non-COX-inhibitor sulindac sulfone suggested an important mechanistic role for COX inhibition in the selective inhibition of Aβ₄₂ secretion by NSAIDs.

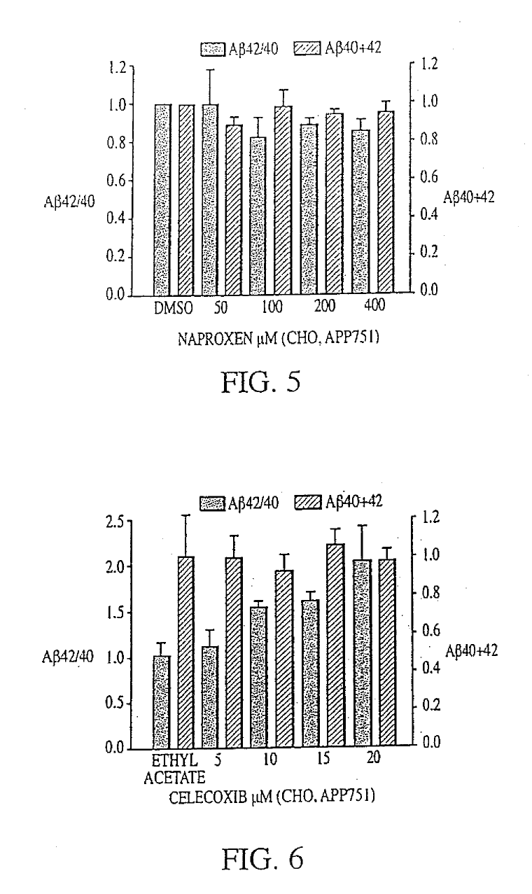

To determine whether reduction of Aβ₄₂ secretion is a common effect of all NSAIDs, other clinically useful NSAIDs were examined. Naproxen is a non-selective COX-inhibitor with an inhibition profile similar to sulindac and a structure similar to ibuprofen (see Cryer et al. (1998) Am J Med 104:413-21). Cell cultures were treated with increasing concentrations of naproxen and aspirin. Aβ₄₀ and Aβ₄₂ levels in cell culture supernatants were analyzed using ELISA. Aβ₄₂/Aβ₄₀ ratios and total Aβ levels were normalized to values obtained from DMSO-treated cultures. Averages of two or three experiments performed in duplicate are summarized in FIG. 5. Treatment of CHO cells expressing APP751 with naproxen, at concentrations up to 400 μM, did not change Aβ₄₂/Aβ₄₀ ratios and did not affect total Aβ levels (see FIG. 5). Similarly, no reductions in Aβ₄₂ secretion were observed when cultures were treated with aspirin concentrations of up to 3 mM (data not shown). Two selective inhibitors of COX-2, celecoxib and rofecoxib, also were examined to determine if they reduced Aβ₄₂ secretion. Celecoxib and rofecoxib were prepared from capsules using solvent extraction and recrystallization. NSAIDs were verified using NMR and mass spectrometry. CHO cells expressing APP751 were treated with various concentrations of celecoxib. FIG. 6 is a bar graph comparing Aβ₄₂/Aβ₄₀ ratios and total Aβ levels in cells treated with ethyl acetate or various concentrations of celecoxib. Results showed that 20 μM celecoxib treatment induced a two-fold increase in Aβ₄₂/Aβ₄₀ ratio. The increase in Aβ₄₂/Aβ₄₀ ratio also was observed when human neuroglioma cells were tested (data not shown). The increase in Aβ₄₂/Aβ₄₀ ratio was not seen in cells treated with rofecoxib at 20 μM (data not shown). Diclofenac and NS-398, two other NSAIDs having preferential activities against COX-2, did not affect Aβ₄₂/Aβ₄₀ ratios or total Aβ levels. Table 1 summarizes selective and non-selective COX-inhibitors that were tested and results of these tests. Reduction of Aβ₄₂ secretion was not associated with all NSAIDs. (Note: peak NSAID concentrations used in these experiments were higher than what was required for complete inhibition of COX-1 and COX-2 activities in in vitro cell-based assays.)

TABLE 1
Non-selective and selective COX-inhibitors tested for effect on Aβ42 levels

| | | | | |
|---|---|---|---|---|
| | | | | COX-1/COX-2 |
| | | | | selectivity# |
| Drug | Highest conc. tested | Plasma | Aβ42/Aβ40 ratio | (1 = equal activity) |

Non-selective COX-inhibitors

| | | | | |
|---|---|---|---|---|
| Sulindac sulfide | 100 (μM) | 14.6 (μM) | selective decrease in Aβ42 | 0.61 |
| Indomethacin | 150 | 1.4 | selective decrease in Aβ42 | 22-58 |
| Ibuprofen | 750 | 40-111 | selective decrease in Aβ42 | 1.69 |
| Naproxen | 400 | 1.3 | no effect | 1.79 |
| Aspirin | 3000 | 111 | no effect | 166 |
| Meloxicam | 100 | 15 | no effect | .01-0.3 |
| Diclofenac* | 600 | 6.1 | no effect | .69 |

Selective COX-2 inhibitors

| | | | | |
|---|---|---|---|---|
| NS-398* | 20 | | no effect | .07 |
| Celecoxib | 20 (μM) | 15 (nM) | selective increase in Aβ42 | .003 |
| Rofecoxib* | 20 (μM) | 3 (nM) | no effect | .001 |

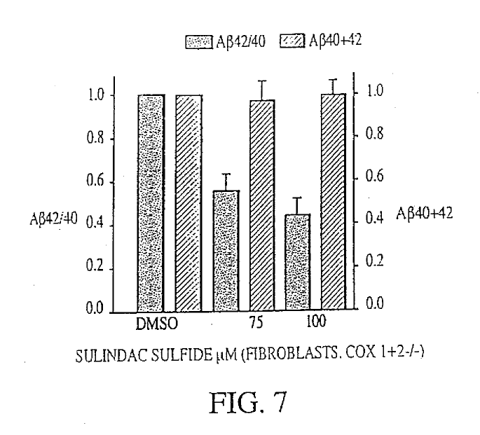

To confirm that NSAID did not reduce Aβ₄₂ secretion through COX inhibition and though reduction of prostaglandin synthesis, cells devoid of COX-1 and COX-2 activities were treated with sulindac sulfide, and Aβ₄₂/Aβ₄₀ ratios were examined. Primary fibroblasts derived from COX-1/COX-2 double-knockout mice, described by Zhang et al. (1999) J Exp Med 190:451-59, were infected with an adenovirus vector that encoded APP695 (see Yuan et al. (1999) J Neurosci Methods 88: 45-54). Fibroblasts infected with the adenovirus vector expressing APP695 were treated with increasing concentrations of sulindac sulfide. Levels of Aβ forms in fibroblast culture supernatants were quantified using ELISA and results are summarized in FIG. 7. (Aβ₄₂/Aβ₄₀ ratios and total Aβ levels were normalized to values obtained from DMSO-treated cells. Results were the averages of two or three experiments, each performed in duplicate.) Sulindac sulfide reduced Aβ₄₂ secretion as well as the Aβ₄₂/Aβ₄₀ ratio of fibroblasts in a fashion similar to that seen with CHO and HS683 neuroglioma cells. Therefore, selective reduction of Aβ₄₂ was not mediated by COX inhibition.

### Example 11

### APP Processing by γ and β-Secretases, APP Turnover, and Notch Intramembrane Cleavage are Not Affected by Sulindac Sulfide

NSAIDs are the only compounds reported so far that change Aβ₄₂/Aβ₄₀ ratios by selectively decreasing Aβ₄₂ secretion. To determine if APP processing and notch intramembrane cleavage were affected in cells treated with NSAIDs, the following experiments were performed.

CHO cell cultures expressing APP751 were treated with increasing concentrations of sulindac sulfide. Cell lysates were prepared, and steady-state APP levels were examined using 4-12% gradient-gel electrophoresis and western blotting using the polyclonal antibody CT15. When western blot analysis was performed, neither a change in APP levels, nor an increase in CTF levels was observed in response to 60 TM or 80 TM sulindac sulfide treatment compared with levels observed for cells treated with DMSO. Unlike published γ-secretase inhibitors, sulindac sulfide did not induce detectable accumulation of APP CTFs. Therefore, β-secretase cleavage was not significantly affected by sulindac sulfide.

When western blot analysis was performed to detect soluble APP (sAPP) in culture supernatants using 5A3/IG7 monoclonal antibodies, results showed that there was no significant change in secretion of the APP ectodomain, (i.e., sAPP), in response to increasing concentrations of sulindac sulfide. Therefore, α-secretase cleavage was not significantly affected by sulindac sulfide.

APP turnover in the presence of sulindac sulfide was examined by (1) pulse labeling CHO cells with ³⁵S-methionine and (2) determination of APP half-life. All values were normalized to a signal obtained at the end of pulse labeling. When the APP half-life in cells treated with DMSO was compared with APP half-life in cells treated with sulindac sulfide at 25 or 125 μM, APP half-life after treatment with 25 or 125 μM sulindac sulfide was similar to APP half-life after treatment with DMSO. Therefore, APP turnover was not altered significantly in the presence of sulindac sulfide.

A significant fraction of Aβ is produced and released in the endocytic pathway after internalization of APP from the cell surface (see Koo et al. (1994) J Biol Chem 269:17386-9). The effect of sulindac sulfide on this endocytic pathway was examined with an APP internalization assay described by Koo et al. (1996) J Cell Sci 109:991-8. APP internalization was expressed as a ratio of cell surface APP versus internalized APP. When APP internalization in cultures treated with DMSO was compared with APP internalization in cultures treated with sulindac sulfide at 60 or 80 μM, the ratio of cell surface APP to internalized APP was not altered in cells treated with sulindac sulfide compared to cells treated with DMSO alone. Therefore, it was concluded that APP internalization was unchanged after sulindac sulfide treatment.

Notch intramembrane cleavage and formation of NICD were analyzed in kidney HEK293 cells. The myc-tagged NotchΔEMV construct encoding a constitutively cleaved Notch variant was transiently transfected into HEK293 cells. Cell cultures were treated with 125 μM sulindac sulfide for 36 hours. Then they were pulse labeled with ³⁵S-methionine for thirty minutes and chased for two hours. Cell lysates were prepared and subjected to immunoprecipitation with monoclonal antibody 9E10. Immunoprecipitated proteins were subjected to SDS-PAGE and phosphor imaging analyses. When amounts of NICD immunoprecipitated from lysates of cells treated with DMSO were compared with amounts immunoprecipitated from lysates of cells treated with sulindac sulfide, results showed that treatment with sulindac sulfide did not impair Notch cleavage and NICD formation. (Cells transfected with a construct encoding only the NICD domain were used for identification of the cleavage fragment.) Similarly, treatment with 500 μM ibuprofen or 150 μM indomethacin did not cause accumulation of APP-CTFs or inhibition of Notch cleavage (data not shown). Overall, these results demonstrated that NSAID treatment did not significantly perturb APP processing or γ-secretase activity. This, however, did not rule out modulation of γ-secretase activity as a mechanism of action for NSAIDs. The selective reduction in Aβ₄₂ secretion could be reflected only in minor changes of γ-secretase activity that may not be detectable in the assays described above.

### Example 12

### Reduction in Aβ₄₂ Secretion was Accompanied by a Dose-Dependent Increase in Aβ₁₋₃₈ Species

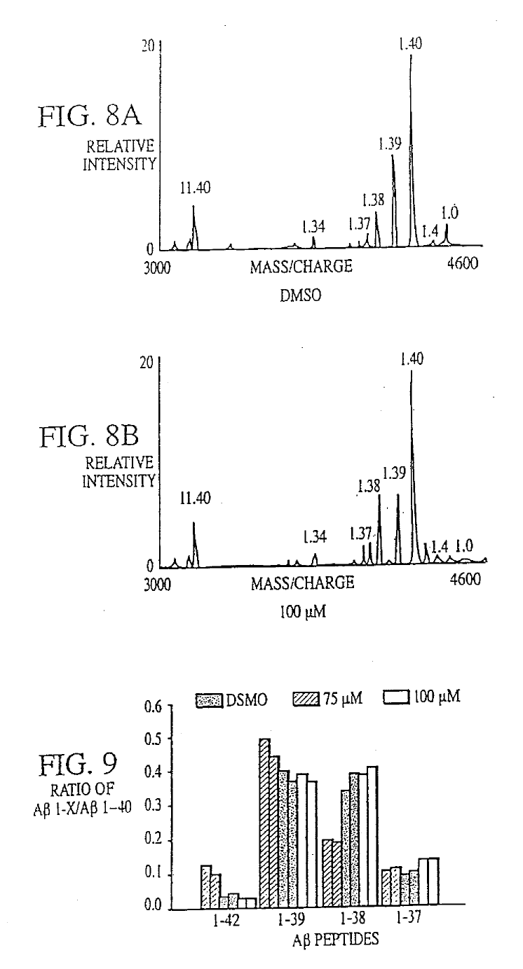

To examine Aβ species secreted by cells treated with sulindac sulfide, immunoprecipitation and mass spectrometry analyses were performed. FIG. 8 is two representative mass spectra of Aβ species secreted by CHO cells expressing APP751 after treatment with DMSO or after treatment with 100 μM sulindac sulfide. After treatment with 75-100 μM sulindac sulfide, a strong reduction in Aβ₄₂ secretion was observed. Levels of Aβ₄₀, however, were largely unaffected. Various Aβ species including Aβ₁₋₄₂, Aβ₁₋₃₉, Aβ₁₋₃₈, and Aβ₁₋₃₇ were quantified. FIG. 9 is a bar graph comparing ratios of each of these species to Aβ₁₋₄₀, i.e. A₁₋ₓ/Aβ1-40 ratios, at 75 or 100 μM sulindac sulfide. Duplicate measurements were used in generating the bar graph. Reductions in Aβ₄₂/Aβ₄₀ ratios were accompanied by two-fold increases in Aβ₁₋₃₈/Aβ₁₋₄₀ ratios. Increases in Aβ₁₋₃₈ levels were dose-dependent. Other Aβ peptide levels did not vary consistently between cells treated with DMSO or with sulindac sulfide.

Mass spectrometry results demonstrating reductions in Aβ₄₂ secretion with concomitant increases in Aβ₁₋₃₈ secretion were confirmed by immunoprecipitation. Aβ polypeptides were immunoprecipitated from culture supernatants of CHO cells expressing APP751 and mutant PS-1. Immunoprecipitates were separated on an SDS-urea gel system that can resolve individual Aβ species (see Wiltfang et al. (1997) Electrophoresis 18:527-32). Standard Aβ₁₋₃₈, Aβ₁₋₄₀, and Aβ₁₋₄₂ peptides were included for identification of different Aβ species. When changes in Aβ₃₈, Aβ₄₀, and Aβ₄₂ levels in CHO cells treated with DMSO were compared with those in cells treated with 60 μM or 80 μM of sulindac sulfide, a reduction in the intensity of an immuno-reactive band corresponding to Aβ₄₂ was observed. This reduction was matched by an equivalent increase in the intensity of an immuno-reactive band corresponding to Aβ₁₋₃₈.

Two potential mechanisms may explain this unprecedented change in Aβ production after NSAID treatment. Sulindac sulfide could reduce Aβ₄₂ secretion by shifting γ-secretase activity towards production of Aβ₁₋₃₈. Alternatively, it may stimulate a novel proteolytic activity that converts Aβ₄₂ into shorter Aβ species such as Aβ₁₋₃₈.

Koo et al. (1994) J Biol Chem 269:17386-9 and others reported that APP processing in the endocytic pathway leads to the generation and release of both Aβ₄₀ and Aβ₄₂ into culture supernatant. To examine the intracellular pool of Aβ₄₂ in APP mutants that lack the endocytic signal, CHO cells expressing an internalization-deficient APP polypeptide lacking 43 amino acids in the cytoplasmic tail were used (Perez et al. (1999) J Biol Chem 274:18851-6). Levels of cellular and secreted Aβ₄₂ and Aβ₄₀ in cells expressing wild type APP and in cells expressing mutant APP were compared using ELISA. Results indicated that in the absence of the cytoplasmic tail, levels of Aβ₄₀ and Aβ₄₂ secreted by cells expressing mutant APP were diminished compared to cells expressing wild type APP. In addition, in the absence of a cytoplasmic tail, cellular Aβ₄₀ levels were reduced while cellular Aβ₄₂ levels were not reduced.

### Example 13

### NSAID Treatment of Tg2576 Transgenic Mice

NSAIDs were dissolved in an appropriate vehicle. Dimethyl sulfoxide (DMSO), ethanol, and ethyl acetate are some examples. The NSAID solution was mixed with Kool-Aid and administered orally using a medicine dropper. For three days, equal doses were administered every four hours, totaling 50 mg/kg/day. At two hours after the final doses were administered, animals were sacrificed, and SDS soluble Aβ₄₀ and Aβ₄₂ were analyzed using ELISA.

### Example 14

### Treatment of Animals with Ibuprofen Reduces Aβ₄₂ Levels

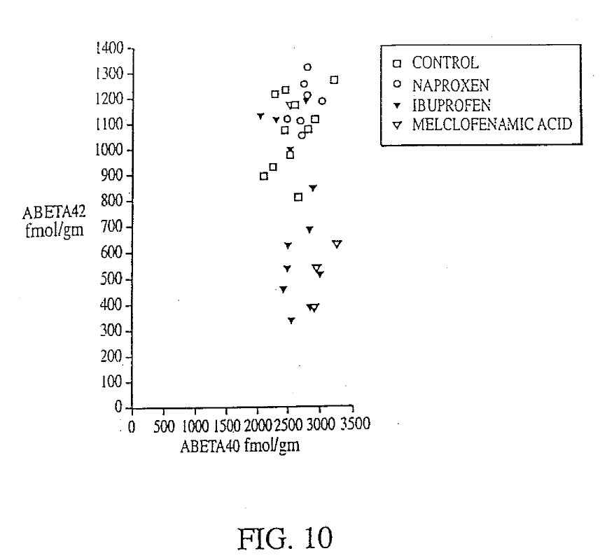

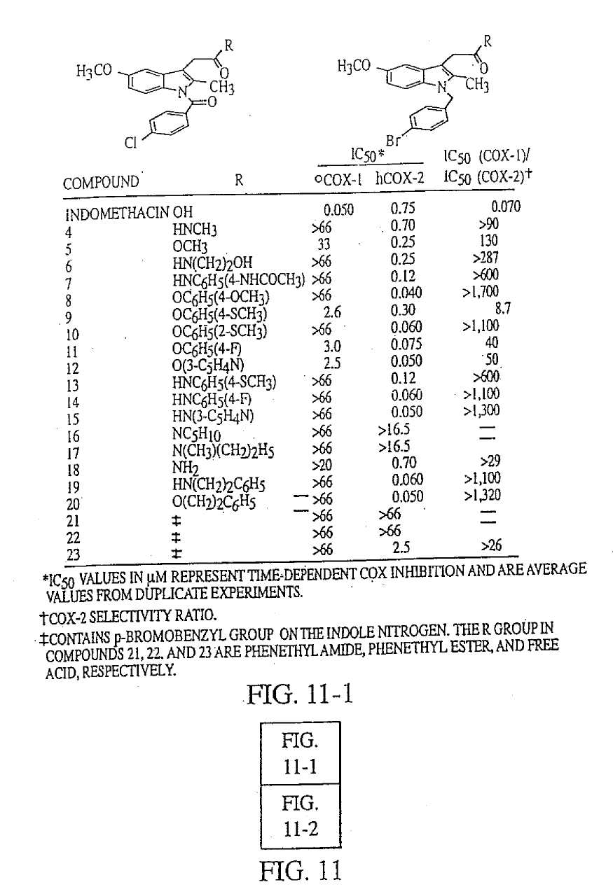

To determine whether acute ibuprofen treatment of mice would reduce Aβ₄₂ levels, three month-old Tg2576 mice expressing APP695 containing the “Swedish” mutation (APP695NL) were used. Three month old mice have high levels of soluble Aβ in the brain but no Aβ deposition (see Hsiao et al (1996) Science 274:99-102). Mice were given naproxen, ibuprofen, or meclofenamic acid as described in Example 13. Mice treated with ibuprofen (n=12) were compared with those untreated (n=11), treated with naproxen (n=7), or treated with meclofenamic acid (n=4). Brain levels of SDS-soluble Aβ₄₀ and Aβ₄₂ were measured using ELISA. Table 2 summarizes Aβ₄₀ and Aβ₄₂ levels determined for the control group and the naproxen, ibuprofen, and meclofenamic acid-treated groups. Treatment with ibuprofen or meclofenamic acid for three days resulted in approximately 30% reduction in Aβ₄₂ levels in the brain, while no change was observed in Aβ₄₀ levels (see FIG. 10). No reduction in Aβ₄₂ levels was observed for naproxen-treated mice. These data were consistent with the rapid onset of Aβ₄₂ reduction in cell culture studies and illustrated that cell culture experiments were able to predict in vivo efficacy. In addition, these data suggested that ibuprofen treatment could prevent amyloid pathology by decreasing Aβ₄₂/Aβ₄₀ ratio in the brain.

TABLE 2
Brain levels of Aβ after acute dosing of Tg2576 mice (mean ± SD)

| | | | | Meclofenamic |
|---|---|---|---|---|
| | Control (n = 11) | Naproxen (n = 7) | Ibuprofen (n = 12) | acid (n = 4) |
| Aβ40 (fmol/gm) | 2603 ± 314 | 2786 ± 179 | 2620 ± 246 | 2932 ± 289 |
| Aβ42 | 1074 ± 145 | 1182 ± 93 | 734 ± 302* | 679 ± 343** |
| % Aβ42 | 29.3 ± 2.9 | 29.8 ± 1.6 | 21.5 ± 7.7* | 18.6 ± 8.7** |

*= p < 0.05;
**= p < 0.01, Dunnett's test

### Example 15

### NSAIDs, NSAID Derivatives, and NSAID Analogues

NSAIDs that are screened for the ability to reduce Aβ₄₂ levels include: FDA-approved NSAIDs, NSAIDs derivatives, and NSAID analogues most potent for reducing Aβ₄₂ levels, newly synthesized derivatives and analogues of the most potent NSAIDs, and NSAIDS known to target pathways other than COX pathways. FDA-approved NSAIDs include ibuprofen, naproxen, dicolfenac, aspirin, indomethacin, fenoprofen, flurbiprofen, ketorolac. Derivatives of the most potent NSAIDs include aryl propionic acid derivatives such as ibuprofen and fenoprofen, and the anthranilic acid derivatives (also called amino carboxylic acid derivatives) such as the meclofenamic acid series and flufenamic acid. (NSAIDs in both series share a similar core structure of either a diphenyl ketone or dephenyl ether.) Other derivatives or analogues that are screened for the ability to reduce Aβ₄₂ levels include flufenamic acid, indomethacin, and meclofenamic acid derivatives and analogues (see FIG. 11 and Kalgutkar et al. (2000) J of Med Chem 43:2860-70). Newly synthesized NSAID derivatives or analogues include novel biphenyl amines (FIG. 12) and diphenyl ketones. Examples of NSAIDs that target additional pathways to COX include LOX inhibitors.

Once a set of NSAIDs, NSAID derivatives, or NSAID analogues having potent ability to reduce Aβ₄₂ levels is obtained, a pharmacophore search is performed to identify other NSAIDs structurally similar to those in the set. If a large number of candidates are identified, the structurally similar NSAIDs are subjected to a secondary structural screen using a computer-based molecular docking algorithm known as EUDOC. In the second structural screen, crystal structures and COX-1/COX-2 binding pockets are used to identify a subset consisting of NSAIDs structurally similar to those that have potent ability to reduce Aβ₄₂ levels but do not bind COX-1 or COX-2. NSAIDs predicted to bind to COX and those predicted to not bind to COX are used as controls.

NSAIDs, NAID derivatives, and NSAID analogues can be obtained commercially or they can be chemically synthesized. Novel NSAIDs, NSAID derivatives, or NSAID analogues with unknown effects on COX activity are tested using in vitro COX-1 and COX-2 assays to determine if there is an affect on COX activity. Commercially available kits from Oxford biochemicals are used for COX inhibition assays.

### Example 16

### Determination of Optimal Screening Interval for Detecting Selective Reduction of β42 Levels

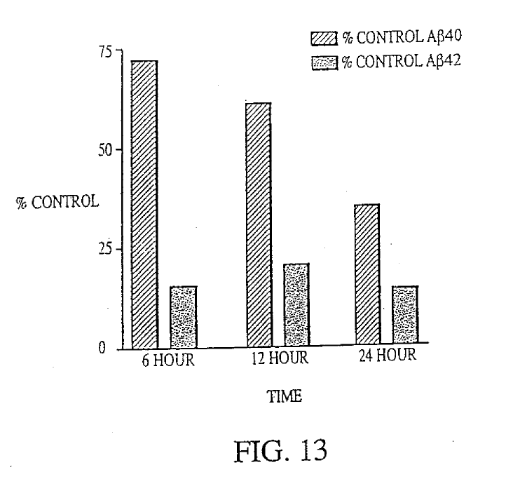

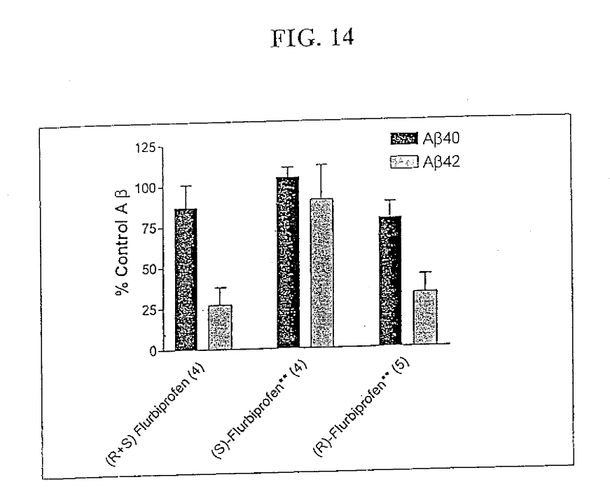

To determine the optimal treatment interval for examining selective reduction of Aβ₄₂ levels, CHO-APP695NL,I,his cell cultures were treated with the vehicle, or treated with ibuprofen or meclofenamic acid for six, twelve, or twenty-four hours. Aβ₄₀ and Aβ₄₂ levels in culture supernatants were determined for each time points using ELISA. FIG. 13 is a bar graph demonstrating that selective reduction of Aβ₄₂ was detectable at six hours when cells were treated with meclofenamic acid. Similar results were observed for ibuprofen (data not shown).

### Example 17

### Primary in vitro Screening

In a primary screen, the effects of NSAIDs on Aβ₄₂ secretion by a CHO cell line that expressed APP (CHO-APP695NL,I,his) were examined. Duplicate cell cultures were treated with (a) a vehicle, (b) 10 μM of NSAID, or (c) 100 μM of NSAID.

To determine Aβ₄₀ and Aβ₄₂ levels, six-hour culture supernatants taken from cells grown in a single well of a twenty-four-well plate were used in end-specific Aβ₄₀ and Aβ₄₂ ELISAs (Suzuki, et al. (1994) Sci 264:336-1340). Aβ₄₀ and Aβ₄₂ levels of cultures treated with NSAID were compared with those of cultures treated with the vehicle alone. Concentrations of 100 μM Ibuprofen and 10 μM meclofenamic acid were used as positive controls. Results, in Table 3, indicated that some NSAIDs selectively reduce Aβ₄₂ levels, but at the concentrations tested, many do not. NSAIDs were classified based on a 20% change in Aβ levels observed in NSAID-treated versus vehicle treated cells. Classification was made based on a 20% change because the data showed a 10% accuracy variance. When classification was made based on a 20% change, all NSAIDs screened, with the exception of two, were classified in the same category with repeated testing. Two NSAIDs, shown in bold italic, gave results that altered their categorization upon re-screening; classification was resolved after a third test. These results confirmed the data described in Examples 8-10, as the NSAIDs that were shown to selectively lower Aβ₄₂ initially also reduced Aβ₄₂ in this screen. Of the newly synthesized biphenyl amines, meclofenamic, mefenamic, and flufenamic acid selectively reduced Aβ₄₂ levels, while tolfenamic acid did not. NSAIDs that caused either selective reduction of Aβ₄₂ levels or reduction in both Aβ₄₀ and Aβ₄₂ levels are subjected to a secondary screen.

TABLE 3
Effects of NSAIDs on secreted Aβ.

| | | % | % | % |
|---|---|---|---|---|
| | | Control | Control | Control |
| Compound | Type | Aβ40 | Aβ42 | % Aβ42 |
| ↓Aβ42 no effect on Aβ40 | | | | |
| Sulindac Sulfide 10 μM | Cox-1,2 | 97% | 57% | 65% |
| Flufenamic Acid 10 μM | Cox-1,2 | 99% | 64% | 70% |
| Ibuprofen 100 μM | Cox-1,2 | 95% | 74% | 81% |
| Ibuprofen 10 μM | Cox-1,2 | 102% | 80% | 82% |
| Flurbiprofen 100 μM | Cox-1,2 | 93% | 70% | 80% |
| Fenoprofen 100 μM | Cox-1,2 | 102% | 60% | 63% |
| Mefenamic Acid 100 μM | Cox-1,2 | 116% | 78% | 72% |
| Indomethacin 100 μM | Cox-1,2 | 101% | 69% | 68% |
| ↓Aβ42 > ↓Aβ40 | | | | |
| NPPB 10 μM | Cox-1,2 | 81% | 48% | 66% |
| Carprofen 100 μM | Cox-1,2 | 58% | 48% | 86% |
| Meclofenamic Acid 10 μM | Cox-1,2 | 39% | 13% | 37% |
| ↓Aβ40 no effect on Aβ42 | | | | |
| APHS 10 μM | Cox2 > Cox1 | 50% | 114% | 178% |
| Resveratrol 10 μM | Cox-1 | 75% | 107% | 130% |
| ↓Aβ40 and ↑Aβ42 | | | | |
| Meloxicam 10 μM | Cox-1,2 | 64% | 122% | 158% |
| SC560 10 μM | Cox-1 > Cox-2 | 47% | 166% | 227% |
| Guaiazulene 100 μM | Cox-1,2 | 70% | 124% | 156% |
| ↑Aβ42 | | | | |
| NS398; 10 μM | Cox-2 > Cox-1 | 101% | 146% | 132% |
| Ketorlolac 10 μM | Cox-1,2 | 84% | 131% | 142% |
| Benzydamine 100 μM | Cox-1,2 | 90% | 128% | 132% |
| ↑Aβ40 and/or ↑Aβ42 | | | | |
| Suprofen 100 μM | Cox-1,2 | 126% | 129% | 102% |
| Indoprofen 100 μM | Cox-1,2 | 116% | 126% | 107% |
| Nabumetone 100 μM | Cox-1,2 | 157% | 103% | 70% |
| Piroxicam 100 μM | Cox-1,2 | 142% | 101% | 75% |
| No Effect on Aβ | | | | |
| Acetylsalicylic acid 100 μM | Cox-1 > Cox-2 | 93% | 99% | 104% |
| Ketoprofen 100 μM | Cox-1,2 | 88% | 107% | 117% |
| Fenbufen 100 μM | Cox-1,2 | 100% | 109% | 107% |
| Naproxen 100 μM | Cox-1,2 | 107% | 112% | 104% |
| Isoixicam 100 uM | Cox-1,2 | 109% | 112% | 103% |
| Tenoxicam 100 μM | Cox-1,2 | 80% | 92% | 112% |
| Tolfenamic Acid 100 μM | Cox-1,2 | 84% | 95% | 110% |
| Diclofenac; 100 μM | Cox-1,2 | 88% | 87% | 100% |
| Etodolac 100 μM | Cox-1,2 | 85% | 109% | 120% |
| Acemetacin 100 μM | Cox-1,2 | 110% | 101% | 93% |
| Niflumic Acid | Cox-1,2 | 120% | 107% | 85% |
| Dapsone | Anti-Bacterial | 99% | 80% | 84% |
| Sulindac Sulfone | No-Cox | 109% | 97% | 92% |
| Nimesulide | Cox-1,2 | 105% | 116% | 116% |
| Suxibuzone | Cox-1,2 | 82% | 107% | 129% |
| Diflunisal | Cox-1,2 | 90% | 103% | 112% |

### Example 18

### Secondary and Tertiary in vitro NSAID Screening

In a secondary screen, an extended dose-response study in which CHO cell cultures are treated with 1 nM to 1 mM of NSAID is performed. Dose response studies are used to estimate IC₅₀ values for maximum reduction of Aβ levels as well as to identify NSAIDs that have toxic effects. A secondary screen is performed for all FDA-approved NSAIDs that reduce Aβ₄₂ levels in cell cultures.

In a tertiary screen, Aβ production, sAPP production, and toxicity in a human H4 neuroglioma cell line that expressed APP are examined for all FDA-approved NSAIDs and novel NSAIDs that selectively reduce Aβ₄₂ levels. Three doses of each NSAID are tested. The first is a dose that is expected to cause maximum reduction of Aβ₄₂ levels. The second dose is one that reduces Aβ₄₂ levels by 50% of the maximum value, while the third dose is one that reduces Aβ₄₂ levels by 10-20% of the maximum value. Tertiary screens are performed on the most potent NSAIDs identified by secondary screens.

NSAID toxicity is measured using an MTS assay (see Example 1) and a lactate dehydrogenase (LDH) release assay (Promega Corp, Madison, Wis.).

### Example 19

### Acute Single-Dose Studies to Identify NSAIDs Having in vivo Activity

To determine whether NSAIDs that selectively reduce SDS-soluble Aβ₄₂ levels in cell culture studies also reduce brain Aβ₄₂ levels, in vivo studies using Tg2576 mice are performed.

Mice of either sex are used for acute studies. Each experimental group, however, is performed using mice of the same sex. Power calculations, based on past measurements of variability of Tg2576 brain Aβ levels, indicate that an “n” of five mice per study group gives an 80% chance of detecting a difference of 20% or more at p<0.05. These calculations are supported by experiments on wortmanin treated and Aβ₄₂ immunized Tg2576 mice, in which significant changes in Aβ levels, even between groups of three to four mice, were noted (Haugabook et al. (2000) Faseb J). Although in most studies there are five mice per experimental group, in some instances, additional mice are used to account for loss due to death or illness. The use of additional mice also increases the power of ancillary studies such as those involving behavior, as sometimes, the number of mice needed to obtain a useful result is not known.

NSAIDs are prepared and administered to three-month-old Tg2576 mice as described in Example 13. To avoid extensive testing of NSAIDs that are not active in vivo, high doses of NSAIDs are used initially. NSAIDs are administered every four to eight hours; exact doses and dose schedules are determined from LD₅₀ values, half-lives, and in vitro dose response studies. In general, a maximum dose that is non-toxic, typically ranging from 1/10 to ⅕ of the LD₅₀ value of the NSAID, is used. If the LD₅₀ and other pharmacokinetic data of a given NSAID are unknown, their values are estimated using those of the nearest structural analogue.

To monitor toxicity, weights of a mouse before and after the study are compared. In addition, one mouse from each treatment group is subjected to a liver function test (LFT) in which blood levels of two liver enzymes, SGOT and SGPT, are determined. SGOT and SGPT are sensitive markers of liver toxicity. Furthermore, renal function, indicated by blood urea nitrogen (BUN) levels, is determined. Tests for liver and renal functions are performed by Anilitics (Gaithersburg, Md.), a company that specializes in these tests. Those NSAIDs having toxic effects at high doses are not used in long-term studies unless their effectiveness and lack of toxicity at lower doses are established.

Following a three-day administration schedule, mice are sacrificed; Aβ levels in plasma, brain, and CSF are determined; levels of NSAIDs in plasma are determined; and mice are examined for signs of toxicity. NSAIDs that selectively reduce SDS-soluble Aβ₄₂ levels by more than 20-30% are examined in multiple dose response studies.

### Example 20

### Multiple-Dose Studies to Identify Doses Useful for in vivo Long-Term Animal and Human Studies

NSAIDs that reduce Aβ₄₂ levels in vivo, at high doses, are administered to groups of three mice at high, medium, and low doses using the same dosing regimen described in Example 19. A high dose is the amount used in the single dose screen of Example 19, while medium and low doses are determined by inference from in vitro dose response studies described in Example 18. Those NSAIDs more potent than ibuprofen in vitro, (i.e., those that have IC₅₀ values required for maximum reduction of Aβ₄₂ levels that are less than a mid TM value) are examined over a wide range of doses. For example, doses representing 1/50 to 1/10 of the IC₅₀ value are used in the multiple dose analysis. NSAIDs having similar in vitro IC₅₀ values to ibuprofen are tested over a more limited range. For example, doses representing 1/10 to ⅓ of the IC₅₀ value are used in the multiple dose analysis. Analyses of Aβ are performed as described for single dose studies. To identify plasma NSAID levels that correlate with Aβ₄₂ reduction in vivo, a plasma NSAID level is determined for each dose examined using the HPLC method described in reference 64 and adapted for each particular NSAID. Data pertaining to plasma NSAID levels in these multiple dose studies are used as reference values for both long-term animal studies where NSAIDs are administered in feed, as well as for subsequent human studies.

### Example 21

### Effects of NSAIDs on in vivo COX Activity

To determine if concentrations of NSAIDs used are sufficient to mediate anti-inflammatory effects, novel NSAIDs are examined for their in vivo COX inhibitory activities and anti-inflammatory activities. For this study, the carrageneenan-induced footpad edema assay, described in Kalgutkar et al. (2000) J of Med Chem 43:2860-70, is performed on mice prior to sacrifice. For NSAIDs that do not reduce Aβ₄₂ levels, the assays are performed on mice treated with NSAIDs at levels equivalent to that administered in long-term studies.

### Example 22

### NSAIDs Used in Long-Term Preventative and Therapeutic Studies

To determine whether the effects of NSAIDs on amyloid deposition in an animal model are attributable to direct inhibition of Aβ₄₂ accumulation, or reduction in inflammatory processes in the brain, or both, the following groups of NSAIDs are examined in long-term preventative and therapeutic tests. NSAIDs that selectively reduce Aβ₄₂ levels but lack anti-inflammatory properties, NSAIDs that selectively reduce Aβ₄₂ levels and have anti-inflammatory properties, or NSAIDs that have no effect on Aβ₄₂ levels but have anti-inflammatory properties are examined in both preventative and therapeutic studies. Ibuprofen is used to examine indirect inflammatory-mediated effects on Aβ deposition and direct effects caused by reduction of Aβ₄₂ levels, since it reduces Aβ₄₂ levels and has anti-inflammatory properties. Celecoxib and naproxen, non-selective and selective Cox inhibitors, respectively, that do not cause reduction of Aβ₄₂ levels are used to examine Aβ₄₂-independent inflammatory-mediated effects. NSAIDs examined in both preventative and therapeutic studies include those that exhibit one of these three properties: selectivity for Aβ₄₂ reduction relative to COX inhibition, Aβ₄₂ reduction and COX-2 selectivity, or solely increased potency for Aβ₄₂ reduction in vivo.

### Example 23

### Long-Term NSAID Dosing for Preventative and Therapeutic Trials

Long-term dosing of mice is achieved through feed. Feed containing the desired concentration of NSAID can be obtained from commercial entities. Prior to long-term preventative or therapeutic studies, successful administration of a chosen dose of NSAID through feed is verified using the following experiment. First, an NSAID concentration effective in reducing Aβ₄₂ levels in acute studies, when administered by dropper, is chosen. This concentration corresponds to the lowest dose that can generate a maximum reduction in Aβ₄₂ levels. In the case of an NSAID that does not reduce Aβ₄₂ levels, a concentration sufficient to cause anti-inflammatory effects is chosen. In the case of ibuprofen, the dose that reduces Aβ₄₂ levels also is a dose that causes anti-inflammatory effects. Feed containing the chosen concentration of NSAID is used in a short-term trial to compare mice given NSAID by dropper to mice given NSAID incorporated into feed. The reduction in Aβ₄₂ levels as well as peak plasma levels of NSAID are determined for mice given NSAID by dropper and mice given NSAID through feed. If levels of Aβ₄₂ reduction and peak plasma levels of NSAID in the two groups are comparable, then the chosen amount of NSAID is achieved through feeding, and long-term preventative or therapeutic studies are performed. If levels of Aβ₄₂ reduction and peak plasma levels of NSAID in the two groups are not comparable, then the concentrations of NSAID in feeds are altered appropriately until reduction in Aβ₄₂ levels and peak plasma levels of NSAID in the two groups of mice are comparable.

### Example 24

### Determination of Peak Plasma Levels of NSAIDs

Techniques for determination of ibuprofen, fenoprofen, and meclofenamic acid levels in plasma are described in Canaparo et al. (2000) Biomedical Chromatography 14:219-26; and Koup et al. (1990) Biopharmaceutics & Drug Disposition 11: 1-15. In general, an internal standard is added to a plasma sample. The sample is acidified and subjected to organic solvent extraction. The organic phase is dried, dissolved in a small volume, and subjected to HPLC using a C18 column. Calibration and standardization are carried out using untreated plasma spiked with NSAID for construction of a calibration curve.

### Example 25

### CSF Collection

Mice are anesthetized with pentobarbital (30-50 mg/kg). An incision from the top of the skull to the mid-back is made and the musculature from the base of the skull to the first vertebrae is removed to expose the meninges overlying the cisterna magna. The animal is placed on a narrow platform in an inverted fashion beneath a dissecting microscope. The tissue above the cisterna magna is excised with care not to puncture the translucent meninges. The surrounding area is cleaned gently with the use of cotton swabs to remove any residual blood or other interstitial fluid. The dilated cisterna magna containing CSF is easily visible at this point. In addition to the cerebellum, brain stem, and spinal cord, an extensive vascular network also is visible. A micro needle and a polypropylene narrow bore pipette are aligned just above the meninges. With care not to disrupt any of the underlying vasculature, the micro needle is slowly inserted into the cistern. The CSF, which is under a positive pressure due to blood pressure, respiration, and positioning of the animal, begins to flow out of the needle entry site once the micro needle is removed. The micro needle then is pulled slowly backwards and the narrow bore pipette is used to collect the CSF as it exits the compartment. Once the needle is completely removed, the pipette is lowered into the puncture site and used to remove any remaining CSF. The primary collection usually takes less than 15 seconds for completion. The cistern will refill with several TL of CSF within two minutes. A second collection is performed to increase the net yield. At the end of the procedure, the emptied cistern is collapsed due to the removal of CSF. CSF is not collected past the first two minutes. The isolated CSF is transferred quickly into a pre-chilled polypropylene tube on ice. Less than 5% of samples contain visible blood contamination.

### Example 26

### Biochemical, Histochemical, Behavioral, and Toxicology Evaluations of Long-Term NSAID Treatment

When mice are sacrificed, one hemi-brain is processed for biochemical analyses and the other for immunohistochemical and histochemical analyses.

Aβ₄₀, Aβ₄₂, and total Aβ levels in mice brains are determined. Both SDS-soluble and SDS-insoluble formic acid-soluble fractions are examined. ELISA, described in Kawarabayashi et al. (2001) J. Neur 21:372-381, and the BAN50 system, described in Suzuki et al. (1994) Sci 264: 1336-1340, are used. Both Aβ₄₀ and Aβ₄₂ polyclonal capture antibodies and end-specific polyclonal antibodies are available. Changes in levels of different Aβ species due to NSAID treatments are examined by imunoprecipitation-mass spectral analysis. Aβ levels in plasma and CSF are determined at the time of sacrifice.

To examine total plaque burden, brain sections are stained with anti-Aβ antibodies. Antibodies to all Aβ species as well as end specific Aβ₄₀ and Aβ₄₂ antibodies are used. Cored plaques are detected by staining with thioflavin. Plaque number and amyloid burden are calculated as described in the Sigma ScanPro image analysis software (see Haugabook et al. (2000) Faseb J). Plaque types and extent of vascular and parenchymal amyloid depositions are examined.

Inflammation is examined by biochemical and histochemcial techniques. Astrocytosis is examined using immunohistochemical staining and Western blotting of the SDS-extract for GFAP. Microglial activation is examined using staining techniques for anti-phophotyrosine as described in Lim et al. (2000) J Neurosci 20:5709-14. Alternatively, microglia are immunostained using a pan MHC antibody or using SMI-312 GS lectin as described in Frautschy et al. (1998) Am J of Path 152:307-17. Inflammatory markers such as α1ACT and APOE are examined using Western blot analysis of the SDS-extract, while IL-1 and IL-6 are examined using commercially available ELISA kits.

To examine neuronal loss and tau pathology, sections from brains are stained using haematoxylon and eosin. Sections are examined for overt pathological signs and neuronal loss. Marked neuronal loss is quantitated using stereological counting. Tau pathology is assessed using immunohistochemical staining by several anti-phosphorylated tau antibodies.

For behavioral studies, a modified version of the Morris watermaze is used to detect learning and memory impairments related to amyloidosis in mice over-expressing APP (see Chen et al. (2000) Nature 408:975-979). Testing is conducted in fully counterbalanced, age-matched squads of mice (five to seven per group); trial blocks are run at the same time each day, during the light cycle. Subjects run in a fixed order each day with an inter-trial interval of approximately fifteen minutes. Trial spacing minimizes effects of hypothermia and fatigue that often are seen in older animals (see Rick et al. (1996) J Gerontol A Biol Sci Med Sci 51 :B253-60). The first day of testing consists of swimming to a visible platform. This assesses motivation, and visual and swimming ability. One trial is performed from a fixed starting position to each of four separate cued platform locations. In subsequent days, up to ten trials per day are performed using a learning criterion of three consecutive trials with less than twenty escape latency (see Chen et al. (2000) Nature 408:975-979). No probe trial is necessary since the only dependent variable measured is trials to reach criterion (TTC). Once an animal reaches criterion on one platform location, it is immediately switched to a new location. Testing is continued until five platform locations have been learned. Deficits in TTC are apparent in this paradigm primarily on the last two platform locations. These data are used with neuropathological data to assess the mice (see Chen et al. (2000) Nature 408 :975-979).

Evaluation of neurological and sensorimotor skills is performed on the first day of testing, before the cued platform trial. A standard test battery is administered. This consists of (a) ten minutes in an automated open field, (b) examination of righting and grasping reflexes, (c) latency to fall when suspended from a wire by the forepaws, and (d) rotorod performance. These tests screen basic functions such as strength, balance, and locomotor/exploratory behavior that can affect watermaze performance (Rick et al. (1996) J Gerontol A Biol Sci Med Sci 51: B253-60; Murphy et al. (1995) Neur Learn Mem 64:181-6; Bickford et al. (1997) Neur Aging 18,309-18; Cammisuli et al. (1997) Behav Brain Res 89:179-90; and Lewis et al. (2000) Nat Genet 25:402-5). In this way, effects of strength, balance, and locomotor/exploratory behavior on watermaze performance are accounted for.

As in acute studies, appropriate plasma markers are tested intermittently on a few NSAID treated mice to monitor liver and renal functions in both preventative and therapeutic trails. Weights of the mice are monitored bi-weekly, and complete blood counts are performed every two to three months. At the time of sacrifice, the GI tract is examined for signs of ulceration using a dissecting microscope as described in Kalgutkar et al. (2000) J of Med Chem 43:2860-70.

### Example 27

### Determination of the Effects of NSAIDs on Aβ Deposition—Long-Term Preventative Trial

NSAIDs that selectively reduce Aβ₄₂ levels in acute studies are examined in a preventative trial to determine if they can prevent Aβ deposits. Six-month-old Tg2576 mice are used in preventative trials since Aβ deposit has not yet taken place. NSAID treatment of mice at this age corresponds to treating humans before signs of clinical disease occur.

Tg2576 mice are treated with experimentally optimized doses of NSAID for three, six, and twelve months. Each treatment group consists of a minimum of twenty animals, five of which are examined at each of the three time points. The remaining five mice are included in case of illness or death during long-term dosing. Three to four mice are placed into a treatment group each month until groups of twenty animals are established. At the time of sacrifice, tissues obtained for analysis are stored until all the mice within an experimental group have been sacrificed. Therefore, all samples from mice within one experimental group are examined simultaneously. For ibuprofen, naproxen, and control groups, twenty-seven mice are used per experimental group. The extra mice are treated for twelve months after which time behavioral patterns and additional pathologic parameters are examined.

The following NSAIDs are used in preventative trials: ibuprofen which reduces Aβ₄₂ levels, has anti-inflammatory activity, and has a short-half life; meclofenamic acid which is more potent at reducing Aβ₄₂ levels in vitro, and has anti-inflammatory activity; sulindac which reduces Aβ₄₂ levels, has anti-inflammatory activity, and has an extended-half-life; naproxen which has no effect on Aβ₄₂ levels, but has anti-inflammatory activity, and COX-1 and COX-2 inhibitory activities; and celecoxib which is an anti-inflammatory COX-2 selective agent. In addition, other NSAIDs that reduce Aβ₄₂ levels but show selectivity for this effect over inhibitory effects on COX-1, COX-2, or both are included in this study. At three, six, and twelve months of treatment with NSAIDs, mice are analyzed for behavioral alterations; then they are sacrificed and biochemical analyses are performed as described in Example 26.

### Example 28

### Alteration of Aβ Deposits by NSAIDs—Long Term Therapeutic Trial

To determine whether Aβ deposition, the effects of Aβ deposition, or both can be altered once Aβ has accumulated to a high level, NSAIDs that selectively reduce Aβ₄₂ levels in acute studies are examined in a therapeutic trial. Effects of treatment with NSAIDs that reduce Aβ₄₂ levels are compared to effects of treatment with NSAIDs that do not reduce Aβ₄₂ levels such as the non-selective COX inhibitor naproxen and the selective COX inhibitor celecoxib. Sixteen-month-old Tg2576 mice are treated with experimentally optimized doses of NSAIDs for three or six months. Sixteen-month-old mice have large amounts of Aβ in the brain and therefore, are representative of human patients with clinical signs of AD. Amyloid deposition, behavior, and AD-like pathology are examined as described in Example 26. Fourteen mice per treatment group are used; at least five treated and five control mice are compared.

### Example 29

### Statistical Analysis

Mann-Whitney and Dunnet's tests are used for comparisons between groups of treated and untreated mice. A number of correlative comparisons are made. Variables and outcomes used in statistical analysis for each study are the following. For in vitro screening experiments, variables include: Aβ levels in media, NSAID concentrations, toxicity, and COX inhibitory activity; while primary outcomes include reduction in Aβ₄₂ levels and COX inhibitory activity. In acute single-dose studies, variables include: Aβ levels in brain, plasma, and CSF; NSAID concentrations in plasma and brain; and dose of NSAID. Primary outcomes of acute single-dose studies include reduction in brain Aβ₄₂ levels and plasma levels of NSAID, while secondary outcomes includes correlation of brain, CSF, and plasma Aβ levels. In long-term studies, variables include: Aβ levels in brain, plasma, and CSF; NSAID concentrations in plasma (and brain, if possible); dose of NSAID; amyloid burden; extent of inflammatory response; behavioral performance; and toxicity. Primary outcomes of long-term studies include effects on Aβ levels in the brain, while secondary outcomes include evaluation of inflammatory response, behavior, toxicity, and correlative analyses.

### Example 30

### Clinical Investigations in Amyloid-Reducing Actions of NSAIDs

The most promising FDA-approved NSAIDs, determined by preclinical studies, are examined for amyloid reducing actions in healthy subjects as well as subjects with mild to moderate Alzheimer's disease (AD). These studies are performed in three-group parallel design; each group consists of twelve subjects. Subjects are treated with an NSAID or a matching placebo several times a day, depending on the NSIAD, for fourteen days. Study NSAIDs are purchased and over-encapsulated by the San Diego VAMC Pharmacy service or by another compounding pharmacy. Placeboes are similarly encapsulated.

AD subjects are selected based on the following criteria. Subjects consist of men and women, ages 60-85, who are diagnosed with probable AD using the National Institute of Neurologic Communicative Disorders and Stroke-Alzheimer's Disease and Related Disorders Association (NINCDS-ADRDA) test (McKhann et al. (1984) Neurology 34:939-944) or have mild to moderate dementia as determined by the Mini-Mental State Examination (MMSE, Mohs et al. (1996) Int Psychogeriatr 8:195-203). MMSE scores in the range of 15-25 indicate mild to moderate dementia. AD subjects have caregivers that can ensure compliance with medication regimens and with study visits and procedures.

Non-demented control subjects consist of men and women ages 60-80. Control subjects lack significant cognitive or functional complaints, or depression as determined by the Geriatric Depression Scale (GDS), and have MMSE scores in the range of 27-30. Control subjects have the same general requirements as AD subjects with the exception that caregivers are not required. Both AD subjects and control subjects have good general health, i.e., subjects do not have serious or life-threatening comorbid conditions.

Subjects who have medically active major inflammatory comorbid condition(s) such as rheumatoid arthritis, or those who have peptic ulcer, gastro-intestinal bleeding, or intolerance of NSAIDs in the past are excluded from the study. Those who have contra-indications to lumbar puncture, such as severe lumbar spine degeneration, sepsis in the region of the lumbar spine, or a bleeding disorder are excluded from participation in the study. In addition, subjects who currently or recently use medications such as NSAIDs, prednisone, or immunosuppressive medications such as cyclophosphamide that could interfere with the study are excluded. Recently is defined as within one month before undergoing the baseline visit (see next paragraph). Subjects undergoing acetylcholinsterase inhibitor (AChE-I) treatments for AD are not excluded if these subjects have been on stable doses for at least four weeks. Similarly, AD subjects taking antioxidants such as vitamin E, vitamin C, or Gingko biloba are not excluded if they have been on stable doses for at least four weeks. Subjects who use NSAIDs or aspirin on a regular basis are excluded. If needed, analgesics such as paracetamol (Tylenol) are provided during the fourteen-day study.

The study procedure consists of three in-clinic visits: an initial screening visit, a baseline visit, and a follow-up visit at fourteen days. During the screening visit, information needed to assess eligibility is obtained and MMSE is administered.

During the baseline visit, which takes place within two weeks of the screening visit, physical examinations and lumbar punctures are performed. Blood samples are drawn for laboratory tests such as APO-E genotyping and for plasma preparation (see Example 31). At this time, subjects or caregivers, in the case of AD subjects, are given a fourteen-day supply of study NSAID along with instructions about timing of doses and potential adverse effects. (For AD subjects, caregivers are required to accompany subjects to each visit, and are responsible for monitoring and supervising administration of study NSAIDs.) A calendar is provided on which times of medications and potential adverse symptoms are recorded.

The NSAID treatment regimen consists of a fourteen-day treatment with NSAIDs in the form of capsules taken two or three times a day with meals. A high and a low study dose of NSAID are used. For ibuprofen, study doses of 800 mg and 400 mg are used. A study dose of 800 mg consists of two 400 mg ibuprofen tablets, while a study dose of 400 mg consists of one 400 mg ibuprofen capsule and one placebo capsule. For sulindac, a study dose of 200 mg twice a day for a total of 400 mg per day is used. For meclofenamic acid, study doses of 100 mg and 400 mg per day are tested. NSAIDs are pre-packed into a day-by-day plastic medication dispenser.

During the follow-up visit, twelve or fourteen days after beginning treatment, vital signs and adverse side effects of study NSAIDs are assessed. Surplus NSAIDs are returned and counted. In addition, lumbar punctures are performed and blood samples are drawn for laboratory tests and for plasma preparations.

Visits during which lumbar punctures are performed and blood samples are drawn are scheduled for mornings with overnight fasting to avoid obtaining post-prandial or hyperlipemic plasma samples, which can influence levels of Aβ₄₀ and Aβ₄₂. Table 4 summarizes biological markers that are analyzed from plasma and CSF samples.

TABLE 4
Plasma and CSF biological markers

| | | | Volume |
|---|---|---|---|
| Assay | Method | Volume of CSF | of Plasma |
| Protein, glucose, cells | | 1 mL | |
| Aβ₄₀ | ELISA | 100 TL × 2 (in | 100 TL × 2 |
| | | duplicate) | |
| Aβ₄₂ | ELISA | 100 TL × 2 (in | 100 TL × 2 |
| | | duplicate) | |
| Aβ₃₈ | Mass Spectrometry | 1 mL | |
| Isoprostanes | Gas Chromatography/ | 2 mL | |
| | Mass Spectrometry | | |
| M-CSF | ELISA | 50 TL × 2 (in duplicate) | |
| MCP-1 | ELISA | 50 TL × 2 (in duplicate) | |
| Tau, | ELISA | 50 TL × 2 (in duplicate) | |
| P-tau181 | | 50 TL × 2 (in duplicate) | |
| Plasma levels of NSAIDs | HPLC | | 1 mL |

### Example 31

### Collection of Plasma and CSF

Plasma samples are prepared within 15-30 minutes after blood samples are drawn. Plasma samples are frozen at −70° C. until used. At least 6 mL of CSF and, whenever possible, 10-15 mL are drawn from each subject. Total cell, protein, and glucose estimations are performed. Samples are identified by a study ID number, and technicians who run ELISAs or other assays are blinded to the identity of the subjects or the treatment conditions.

### Example 32

### Specific Assays

ELISA is used to determine Aβ₄₀ and Aβ₄₂ levels in CSF. Batches of samples are assayed simultaneously in duplicate on microplates according to established procedures (3). In Aβ₄₂ detection, two antibodies are used: (1) a monoclonal antibody that recognizes an epitope within the first five amino acids of Aβ is used for capture and (2) an end-specific monoclonal antibody that recognizes Aβ ending at amino acid 42 and conjugated to horse radish peroxidase is used for detection. CSF levels of Aβ₃₈ are measured by mass spectroscopy as described in Example 6. CSF isoprostanes are measured by gas-chromatography/negative chemical ionization mass spectroscopy using internal standards for calibration Montine et al. (1999) Neurology 52:562-565). CSF levels of MCSF, MCP-1, tau, and P-tau181 are determined. Commercially available ELISA kits are used for M-CSF (R&D Diagnostics) and MCP-1 (Pharmingen, San Diego) determinations. CSF tau and P-tau181 are determined using ELISA kits from Innogenetics, Inc., Plasma levels of specific NSAIDs are determined by HPLC methods described in published procedures (Canaparo et al. (2000) Biomed Chromatogr 14:219-26).

### Example 33

### Analysis of Clinical Data

Reduction in Aβ₄₂ levels due to NSAIDs treatment is detected as decreases in Aβ₄₂ levels in CSF and/or plasma. Therefore, subjects with AD or elderly control subjects who receive NSAID treatments show serial decreases in CSF and/or plasma Aβ₄₂ levels, while those who take a placebo will not show serial changes in CSF and/or plasma Aβ₄₂ levels.

To assess comparability between groups of subjects at baseline, demographic data (e.g. age and gender), dementia severity (MMSE score), and APO-E e4 allele frequency are compared between placebo groups, and groups of subjects with AD or elderly controls that are treated with NSAIDs. Continuous variables are compared by ANOVA and frequencies of categorical variables such as gender and APO-E genotype are compared using Chi-squared or Fisher's exact test.

Changes in levels of biomarkers of interest between baseline samples to follow-up samples are calculated for each subject. Descriptive statistics are used to determine whether levels of biomarkers at baseline are normally distributed. If they are, then mean changes in each treatment group are compared with each placebo group using ANOVA. If they are not normal, then data transformation is applied or non-parametric statistics are used to compare changes in biomarker levels between different groups of subjects.

To determine whether changes in Aβ₄₂ levels are accompanied by changes in Aβ₄₀ and Aβ₃₈ levels, CSF Aβ levels in placebo groups are compared to that in treatment groups using ANOVA. Levels of biomarkers related to microglial function (e.g. M-CSF and MCP-1), oxidative damage in the brain (e.g. F-2 isoprostanes), and neuronal degeneration (e.g. tau and P-tau181) are compared before and after treatment as well as between groups treated with placebo or with NSAID. If levels of biological markers change after treatment with NSAID, the change is examined in relation to variables such as age, gender, APO-E genotype, and plasma NSAID levels. Scatter-plots and appropriate statistical comparisons are used.

### Example 34

### Statistical Power Calculations

Published data indicate that CSF Aβ₄₂ levels remain stable on repeated lumbar punctures. The power to detect differences between subjects treated with NSAIDs and subjects treated with placeboes depends on magnitudes of changes in biomarker levels after treatment relative to baseline.

In published longitudinal data for CSF Aβ₄₂ levels in an AD patient group of 53 (see Andreasen et al. (1999) Arch Neurol 56:673-80), baseline CSF Aβ₄₂ level (mean±SD) was 709 ±304 pg/ml and follow-up (10 months later) CSF Aβ₄₂ level was 701 ±309 pg/mL. The correlation between the first and second CSF Aβ₄₂ level was R=0.90. No published longitudinal CSF Aβ₄₂ data are available in healthy subjects. In two studies that included healthy subjects, the values for CSF Aβ₄₂ levels were 1485 ±473 pg/mL (see Galasko et al. (1998) Arch Neurol 55:937-45) and 1678 ±436 pg/mL (see Andreasen et al. (1999) Arch Neurol 56:673-80).

The power calculation uses the following assumptions: (1) levels are stable over time as described in Andreasen et al. (1999) Arch Neurol 56:673-80 and (2) variance of change is similar. The standard deviation is calculated as square root of ((1-correlation)*2*SDˆ2). A pre-post correlation of 0.8 for CSF Aβ₄₂ level is assumed.

If the change in pre-post mean CSF Aβ levels is assumed to be approximately zero in the placebo group, then effect size depends on the mean level of Aβ₄₂ at baseline. For example, for elderly controls, if the mean CSF Aβ₄₂ level is 1485 pg/mL (see Galasko et al. (1998) Arch Neurol 55:937-45), then a 0.25 effect size represents an increase or decrease of the mean by 371 pg/mL due to treatment.

For power calculations, the following are assumed: (1) alpha=0.05, (2) power=0.80, and (3) two-group studies in which equal numbers of subjects exposed to placebo and treatment are used. For power calculations with an effect size of 0.25, a sample size (N) of 11 in each of the two groups is required. With effect size of 0.2, an N of 16 is required in each group.

Twelve subjects per group are used for each study allowing for detection of an effect size of 0.25 or higher. In pre-clinical studies, several NSAIDs (including ibuprofen and meclofenamic acid) reduced Aβ₄₂ levels in supernatants from cultured cells and in brain tissues of transgenic mice by over 25%. In long-term transgenic mouse studies using ibuprofen, reported in (Lim et al. (2000) J Neurosci 20:5709-14), Aβ levels in the brain were about 38% lower when treated than untreated.

If the variance in CSF Aβ₄₂ levels between subjects or on repeated lumbar puncture is greater than in these projections, then sample size is re-assessed and group size is modified as needed. A similar set of calculations using published data on CSF Aβ₄₂ levels in AD patients shows that groups of twelve patients are sufficient to detect a 25% effect size.

Published longitudinal CSF data are available for CSF tau in AD. Sunderland et al. (1999) Biol Psychiatry 46:750-755 studied twenty-nine patients with AD having baseline CSF tau (mean±SD) of 548±355 pg/mL, follow-up CSF tau at twelve months of 557±275 pg/mL, and an R-value of 0.85.

The decision to use twelve subjects per group is derived from Aβ data. Again, assuming CSF tau remains stable and unchanged on average in the absence of treatment, an effect size for a decrease in CSF tau by at least 33% relative to baseline is 183 pg/mL of tau.

In a two-group study design with (1) equal subject numbers receiving placebo and treatment, (2) N=12 per group, and (3) assuming α=0.05, then power is 73% for detecting an effect size of 33% or greater for tau.

With the exception of plasma Aβ levels that remained stable as indicated by preliminary ibuprofen studies, the degree of variation of longitudinal measurements of other biomarkers is not known. Ibuprofen studies in healthy elderly and subjects with mild AD are performed first, then sample sizes are reassessed for all biomarkers measured and necessary changes are incorporated in to other NSAID studies.

### Example 35

### Placebo-Controlled Study of NSAIDs with Aβ-Lowering Actions

A double-blind randomized placebo-controlled study is performed using sixty AD subjects treated with a placebo, ibuprofen, or another FDA-approved NSAID with Aβ-reducing action at a well-tolerated dose for 48 weeks. Specific NSAIDs and doses are selected based on results obtained in Example 30.

Subjects are 50-90 years of age and have diagnoses of probable AD as indicated by the NINCDS-ADRDA test. Subjects have an MMSE range of 15-25, good general health, i.e., no life threatening or major medical illnesses; and caregivers who can supervise medication regimens and provide collateral information. Additional screening criteria are as described in Example 30.

Initially, subjects are assessed for eligibility in a screening visit. MMSEs and physical examinations are performed. Blood samples are obtained for routine laboratory tests. Block randomization is used to assign patients to placebo or active treatment groups. Assignment is determined according to baseline MMSE scores so that dementia severity is similar in the placebo and active treatment groups.

During the baseline visit, scheduled within two weeks of the screening visit, vital signs are assessed, lumbar punctures are performed, and blood samples are drawn for APO-E genotyping and for plasma preparation (see Example 31). CSF levels of Aβ₄₀, Aβ₄₂, isoprostanes, tau, and P-tau as well as plasma levels of Aβ₄₀ and Aβ₄₂ are determined. In addition, cognition is assessed using the Alzheimer's Disease Assessment Scale-cognitive component (ADAS-cog, see Galasko et al. (1997) Alzheimer Dis Assoc Disord 11; Suppl 2:S33-9) and MMSE, while functional ability is assessed using the Alzheimer's Disease Cooperative Study Activities of Daily Living Scale (ADCS-ADL) (see McKhann et al. (1984) Neurology 34:939-944). At this time, caregivers of subjects are given a twelve-week supply of study NSAID along with instructions on timing of doses and potential adverse effects.

At the 12-week visit, vital signs, stool guaiac, and adverse side effects are assessed. Unused NSAID is counted. At the 24-week visit, assessment procedures identical to those of the baseline visit are performed. A count of unused NSAIDs and an inquiry about adverse events are made. At the 36-week visit, assessment procedures identical to the 12-week visit are performed, while at the 48-week visit, assessment procedures identical to those of the baseline visit are performed. A count of unused NSAIDs and inquiry about adverse events are made. Table 5 summarizes the examinations performed at each visit in the study.

TABLE 5
Schedule of events

| | Screen | Baseline | 12 week | 24 week | 36 week | 48 week |
|---|---|---|---|---|---|---|
| Check entry criteria, obtain | X | | | | | |
| consent | | | | | | |
| Screening blood tests | X | | | | | |
| Demographics, medical | X | | | | | |
| history | | | | | | |
| Vital signs | X | X | X | X | X | X |
| Rectal examination, stool | X | | X | X | X | X |
| guaic | | | | | | |
| MMSE | X | X | | X | | X |
| ADAS-cog, ADCS ADL | | X | | X | | X |
| Dispense medications | | X | X | | X | |
| Adverse events, pill count | | | X | X | X | X |
| Lumbar puncture, plasma | | X | | X | | |
| for Aβ | | | | | | |
| Blood drawn for safety | | X | | X | | X |
| laboratory tests | | | | | | |

In addition, each subject/caregiver is interviewed by telephone at 4, 8, 16, and 20 weeks to inquire about continuation in the study, medication usage, and adverse events.

### Example 36

### Statistical Analyses of Placebo-Controlled Studies

Statistical analyses involve the comparison of cognitive (ADAS-cog, MMSE), functional (ADCS-ADL), and biomarker data of subjects before and after treatment. Subjects treated with NSAID for 48 weeks are expected to exhibit less cognitive and functional decline relative to subjects who are treated with placebo. NSAID treatments are expected to associate with improved biomarker indices in CSF and possibly in plasma.

Differences (Δs) between final and initial ADAS-cog and ADCS-ADL scores are referred to as primary outcome measures. Mean Δs for placebo and treatment groups are compared by ANOVA. To control for subjects who fail to complete the study, a Last Observation Carried Forward (LOCF) analysis is performed.

Changes in CSF levels of Aβ₄₂, tau, P-tau181, F-2-isoprostanes, and plasma Aβ₄₂ and Aβ₄₀ are similarly analyzed as outcome measures using ANOVA, or a non-parametric test (e.g. Kiruskal-Wallis) if the data are not normal. Correlations between changes in biomarker measures and in clinical measures at 24 weeks are examined by scatter-plots and correlational analyses.

### Example 37

### Cell Culture Studies of Flurbiprofen

APP695-transfected H4 cells were treated with either the R or S enantiomers of flurbiprofen or with racemic mixtures (R+S) of flurbiprofen. Similar effects on Aβ₄₂ production were observed in APP695-transfected H4 cells as shown in FIG. 14. Aβ₄₂ levels were lowered by 50% in the presence of 100 μM of the racemic mixture. Similarly, Aβ₄₂ levels were lowered by 20% in the presence of 10 μM of R-flurbiprofen, 49% in the presence of 100 μM R-flurbiprofen, and Aβ₄₂ levels were lowered by 58% in the presence of 100 μM S-flurbiprofen. In contrast, levels of Aβ₄₀ production by these cells were not altered when the cells were exposed to the R, S, or racemic mixture of flurbiprofen.

### Example 38

### Acute Dosing in Tg2576 Mice

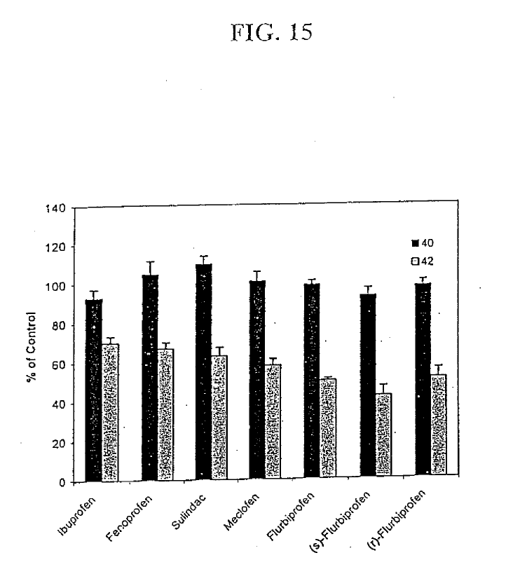

Three-month old Tg2576 mice were treated for three days with 100 mg/kg/day of R-flurbiprofen, S-flurbiprofen, or the racemic mixture (R+S) flurbiprofen. Compounds were administered orally. Levels of Aβ₄₂ in brains of animals treated with flurbiprofen were compared with those treated with vehicle alone. As shown in FIG. 15, R-flurbiprofen was more effective at lowering brain Aβ₄₂ in this acute dosing paradigm than S-flurbiprofen. In contrast, R-flurbiprofen and the racemic mixture had a similar effect on Aβ₄₂ levels in mice brains.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating, or delaying the onset of, Alzheimer's disease in a patient or mild cognitive impairment, comprising:
identifying a patient in need of the treatment or delay; and
administering to the patient of an Aβ₄₂-reducing effective amount of an Aβ₄₂-lowering agent, wherein said Aβ₄₂-lowering agent reduces the level of Aβ₄₂ secretion without substantially affecting the level of Aβ₄₀ secretion from a human cell.

2. The method of claim 1, wherein said Aβ₄₂-lowering agent is an NSAID or a structural derivative or analogue thereof.

3. The method of claim 1, wherein said Aβ₄₂-lowering agent is an NSAID or NSAID structural derivative or analogue chosen from the group consisting of flurbiprofen, fenoprofen, carprofen, meclofenamic acid, flufenamic acid, indomethacin, sulindac sulfide, and structural analogs and derivatives thereof.

4. The method of claim 1, wherein said Aβ₄₂-lowering agent is characterized by the ability to reduce the level of Aβ₄₂ secretion, and increase the level of Aβ₃₈ secretion without substantially affecting the level of Aβ₄₀ secretion in an assay performed in Example 8 or 12 or 17.

5. The method of claim 1, wherein said Aβ₄₂-lowering agent causes at least a 50% reduction in Aβ₄₂/Aβ₄₀ ratio at about 200 μM in the assay of Example 9.

6. The method of claim 1, wherein said Aβ₄₂-lowering agent causes at least an about two-fold increase in Aβ₃₈/Aβ₄₀ ratio at about 75 μM in the assay of Example 12.

7. The method of claim 1, wherein at least one of the following is monitored before and/or after the administration of said Aβ₄₂-lowering agent: (1) the plasma or CSF concentration of Aβ₄₂, (2) the plasma or CSF concentration of Aβ₄₀, (3) the plasma or CSF concentration of Aβ₃₄, Aβ₃₆, Aβ₃₇, or Aβ₃₉, (4) the plasma or CSF concentration of Aβ₃₈, (5) the Aβ₄₂/Aβ₄₀ concentration ratio in plasma or CSF, (6) the concentration ratio between Aβ₄₀ and one of Aβ₃₄, Aβ₃₆, Aβ₃₇, Aβ₃₈ and Aβ₃₉, or a combination thereof, plasma or CSF, (7) the ratio between the concentration of Aβ₄₂ and Aβ₃₈ in plasma or CSF, and (8) the total concentration of Aβ peptides in plasma or CSF.

8. The method of claim 2, wherein said NSAID or NSAID structural derivative or analogue thereof lacks has a reduced the ability to inhibit COX-1, COX-2, or both COX-1 and COX-2 activity.

9. The method of claim 2, wherein said Aβ₄₂-lowering agent is flurbiprofen, or a structural derivative or analogue thereof.

10. The method of claim 1, wherein said Aβ₄₂-lowering agent is a flurbiprofen derivative.

11. The method of claim 1, wherein said Aβ₄₂-lowering agent is a fenoprofen derivative or carprofen derivative.

12. The method of claim 1, wherein said Aβ₄₂-lowering agent is a meclofenamic acid or flufenamic acid derivative.

13. The method of claim 1, wherein said Aβ₄₂-lowering agent is an indomethacin derivative.

14. The method of claim 1, wherein said Aβ₄₂-lowering agent is a sulindac sulfide derivative.

15. The method of claim 1, further comprising administering to the patient an acetylcholinesterase inhibitor.

16. The method of claim 1, further comprising administering to the patient an antioxidant.

17. A pharmaceutical composition comprising:
(a) an Aβ₄₂-reducing effective amount of an Aβ₄₂-lowering agent, wherein said Aβ₄₂-lowering agent reduces the level of Aβ₄₂ secretion without substantially affecting the level of Aβ₄₀ secretion from a human cell;
(b) an acetylcholinesterase inhibitor; and
(c) a pharmaceutically acceptable carrier.

18. A pharmaceutical composition comprising:
(a) an Aβ₄₂-reducing effective amount of an Aβ₄₂-lowering agent, wherein said Aβ₄₂-reducing effective amount of said Aβ₄₂-lowering agent reduces the level of Aβ₄₂ secretion from a human cell;
(b) an antioxidant; and
(c) a pharmaceutically acceptable carrier.
